(19) United States
(12) Patent Application Publication
(10) Pub. No.: US 2019/0064181 A1
(43) Pub. Date: Feb. 28, 2019

(54) IL-22BP AS BIOMARKER IN ANTI-TNF-ALPHA-TREATMENTS

(71) Applicants: Universitaetsklinikum Hamburg-Eppendorf, Hamburg (DE); Yale University, New Haven, CT (US)

(72) Inventors: Richard Flavell, Guilford, CT (US); Samuel Huber, Hamburg (DE); Penelope Pelczar, Hamburg (DE)

(21) Appl. No.: 16/080,063

(22) PCT Filed: Feb. 23, 2017
(86) PCT No.: PCT/EP2017/054218

§ 371 Date: Aug. 27, 2018

(30) Foreign Application Priority Data
Feb. 25, 2016 (DE) 10 2016 103 356.1
Feb. 25, 2016 (LU) 92982

(51) Int. Cl. G01N 33/68; G01N 33/50

(52) U.S. Cl. CPC G01N 33/6869; G01N 33/5082; G01N 33/6893; G01N 2333/525; G01N 2800/52; G01N 2333/54; G01N 2800/065

(57) ABSTRACT

A method for monitoring the effectiveness of an anti-TNF-alpha-treatment, e.g. of an inflammatory bowel disease, using Interleukin-22 binding protein (IL-22BP) as a prognostic biomarker. The method comprises determining the expression level of Interleukin-22 binding protein (IL-22BP) in a biological sample obtained from a subject.

The invention relates to a method for monitoring the effectiveness of an anti-TNF-alpha-treatment, in particular an anti-TNF-alpha-treatment of an inflammatory bowel disease.

Inflammatory bowel disease (IBD) is characterized by chronic intestinal inflammation and dysfunction of the epithelial barrier. The disease initiating primary cause is unclear. Recent findings indicate that alternated interactions between the immune system, epithelial barrier function and microbiome play a crucial role for the development of IBD [1]. It is now well-accepted that inflammatory responses, most likely driven by the microbiome and defective barrier function, promote a vicious circle leading to chronic disease [12]. Since IBD is characterized by chronic inflammation, most therapies are directed against the inflammatory response. Tumor necrosis factor alpha (TNFα) is currently the key target of IBD therapy highlighting its pro-inflammatory properties. Anti-TNF-alpha therapy promotes mucosal healing, although the mechanism is currently unclear. It has been shown, that Interleukin 22 (IL-22) has both protective and pathogenic effects in mouse IBD models [4-8], suggesting that tight control of IL-22 activity is essential in order to maintain tissue protective functions and avoid detrimental effects. IL-22 promotes mucosal healing and is up-regulated in the intestine in patients with IBD [3, 13]. In mouse models it was however shown that IL-22 has also pro-inflammatory functions [7, 8]. Therefore, it is essential to tightly control IL-22 activity. This control is exerted by Inter-leukin-22 binding protein (IL-22BP), which has the same binding site for IL-22 as the membrane-bound Interleukin-22 receptor subunit 1 (IL-22R1). IL-22BP lacks a transmembrane and intracellular domain, however, and is therefore unable to induce signaling. IL-22BP specifically binds IL-22 and prevents binding of IL-22 to membrane-bound IL-22R1 [14-17]. IL-22 has a 20- to 1,000-fold higher binding affinity to IL-22BP compared to its binding to the membrane-bound IL-22R1 [3, 18]. Accordingly, it has previously been shown that endogenous IL-22BP controls IL-22, which uncontrolled can promote tumorigenesis in the intestine [10, 19, 20]. In line with these data, it was shown that transgenic overexpression of IL-22BP in the intestine causes increased colitis susceptibility in a mouse model, in which IL-22 has protective functions [8]. Taken together, these data suggest that a fine-tuned regulation of IL-22 and IL-22BP controls homeostasis in the intestine. Accordingly, it has been reported that IL-22 and IL-22BP exhibit an inverse expression pattern upon tissue damage in the intestine in mouse models: Under steady state conditions IL-22BP is expressed in the colon. It is down regulated locally during intestinal tissue damage and up regulated once again during colonic repair. In contrast, IL-22 expression has an inverse pattern, with maximal expression at the peak of colonic damage and low expression under homeostatic conditions [3, 10, 21]. This regulation of the IL-22-IL-22BP expression pattern is essential to control tissue repair in the intestine in murine models [6, 10].

To date, medical practitioners use clinical symptoms, endoscopic analysis, TNF-alpha antibody levels or the measurement of antibodies against TNF-alpha antibodies for monitoring the therapeutic success of an anti-TNF-alpha therapy. These methods, however, have only limited informative value. It is therefore an object of the invention to provide an improved method for monitoring the effectiveness of an anti-TNF-alpha-treatment of an inflammatory bowel disease.

For this purpose, the invention provides a method for monitoring the effectiveness of an anti-TNF-alpha-treatment in a subject, the method comprising determining the expression level of Interleukin-22 binding protein (IL-22BP) in a biological sample obtained from the subject.

It has surprisingly been found that the expression level of IL-22BP can serve as a prognostic biomarker in IBD. It has been found that the expression level of IL-22BP, in particular the expression level of T-cell derived IL-22BP in inflamed areas of the intestine, positively correlates with TNF alpha expression levels. Without wishing to be bound by theory, it is assumed that, in IBD, TNF alpha causes up-regulation of IL-22BP, which binds to IL-22 and thus prevents mucosal protecting/healing effects of IL-22. Anti-TNFα agents block TNFα, leading to down-regulation of IL-22BP, such that IL-22 is enabled to unfold its healing effects. Therefore the measurement of IL-22BP can serve as a direct biomarker for the effectiveness of anti-TNFalpha treatment.

The term “anti-TNF-alpha-treatment” as used herein means any treatment aimed at directly or indirectly inhibiting the action of tumor necrosis factor alpha (TNFα). Anti-TNFα treatment involves, for example, the use of TNF-alpha inhibitors, i.e. agents directed against TNFα. TNFα inhibitors include, but are not limited to (monoclonal) antibodies binding TNFα. Examples of such antibodies are Infliximab, adalimumab, certolizumab pegol and golimumab. Other examples of TNF-alpha inhibitors are receptor proteins like etanercept, or substances like thalidomide and its derivatives, and xanthine derivatives.

The term “inflammatory bowel disease”, IBD, relates to a group of chronic inflammatory conditions of the gastrointestinal (GI) tract, in particular the large intestine (colon) and the small intestine. IBD primarily includes ulcerative colitis (UC) and Crohn's disease (CD). Both conditions usually involve severe diarrhea, pain, fatigue, vomiting and weight loss. Ulcerative colitis mainly causes long-lasting inflammation and sores (ulcers) in the innermost lining of the large intestine (colon) and rectum. Crohn's disease mainly causes inflammation of the lining of the digestive tract, but can also affect the entire thickness of the bowel wall. It most commonly affects the end of the small bowel (the ileum) and the beginning of the colon. It may, however, affect any part of the GI tract, from the mouth to the anus. In Crohn's disease, the inflammation of the intestine can be “patchy”, i.e. it may leave uninflamed areas in between patches of diseased intestine. Although ulcerative colitis and Crohn's disease account for the most cases of IBD, other conditions are also encompassed by the term. Such other conditions are collagenous colitis, lymphocytic colitis, diversion colitis, Behcet's disease, and indeterminate colitis.

The term “subject” as used herein preferably refers to a vertebrate, further preferred to a mammal, and most preferred to a human.

The term “expression level of Interleukin-22 binding protein” means the quantity of Interleukin-22 binding protein produced in a defined quantity of a biological sample, e.g. a cell suspension or a tissue sample. The expression level may be measured by real-time quantitative reverse transcriptase PCR (qRT-PCR) or on a protein level using ELISA, Western blotting, immune histochemistry, immune fluorescense or flow cytometry. Relative expression levels can e.g. be calculated by methods known in the art (see e.g. [25]).

The term “Interleukin-22 binding protein” (IL-22BP), also known as “cytokine receptor family (CRF) 2-10”, “CRF2-X”, and “IL22RA2”, relates to a soluble IL-22 glycoprotein belonging to the type II cytokine receptor family. IL-22BP lacks a trans-membrane and intracellular domain and specifically binds to IL-22 but not other IL-10 family members, and prevents the binding of IL-22 to membrane bound IL-22R1. As regards human IL-22BP the term in particular relates to its isoform 2 (see e.g. [9]). The term “T-cell derived IL-22BP” relates to IL-22BP produced by T-cells, in particular CD4+CD3+CD11− T-cells.

The term “biological sample” or “biological specimen” as used herein relates to any sample of biological origin or of biological material, e.g. a sample of body fluid, e.g. blood or saliva, feces, a cell sample or tissue sample. The term “biopsy sample” or “biopsy specimen” relates to tissue removed from the body.

The method of the invention may be used to monitor the effectiveness of any anti-TNF-alpha-treatment in a subject subjected to such a treatment. Conditions treated with an anti-TNF-alpha-treatment comprise e.g. psoriasis, rheumatoid arthritis or inflammatory bowel disease (IBD). It is, however, preferred that the condition for which the effectiveness of an anti-TNF-alpha-treatment is monitored, is an inflammatory bowel disease.

For monitoring the effectiveness of any anti-TNF-alpha-treatment it may be sufficient to determine the expression level of Interleukin-22 binding protein (IL-22BP) at any given time during the treatment. The expression level thus determined may, e.g. be compared with accepted standard expression levels for subjects under effective anti-TNF-alpha-treatment or with average expression levels in healthy subjects or subjects in a disease remission state or a state of minimal disease activity. It may thus be sufficient to examine only one sample, without the need for taking any subsequent or previous samples.

In a preferred embodiment of the method of the invention the method comprises determining the expression level of Interleukin-22 binding protein (IL-22BP) in a first biological sample obtained from the subject at a first date and in a second biological sample obtained from the subject at a later second date, and comparing the IL-22BP expression levels at the first and second date. In this embodiment of the method of the invention the expression levels of Interleukin-22 binding protein in a first and a later second biological sample are compared in order to evaluate the effectiveness of an anti-TNF-alpha treatment in a subject, e.g. a subject having IBD. A reduction of the IL-22BP expression level indicates that the treatment is efficient, whereas an essentially unchanged or higher IL-22BP expression level indicates that the treatment is inefficient.

In a preferred embodiment of the method of the invention the first biological sample is obtained at a date before the treatment and the second biological sample is obtained at a date during or after the treatment, or the first biological sample is obtained at a date during the treatment and the second biological sample is obtained at a later date during the treatment or at a date after the treatment. The method of the invention can thus be used to monitor the effectiveness of an anti-TNF-alpha-treatment over time.

In a further preferred embodiment of the method of the invention the biological sample is a biopsy specimen. In case of the condition being treated with an anti-TNF-alpha-treatment being inflammatory bowel disease (IBD), the biopsy specimen is taken from the intestine of the subject, preferably a biopsy specimen taken from an inflamed area of the large intestine or small intestine of the subject.

In a further preferred embodiment of the method of the invention the expression level of T-cell derived IL-22BP is determined. In this embodiment of the invention, cells from the biological specimen, e.g. a biopsy specimen from an inflamed area of an affected organ or tissue, e.g. the intestine, are sorted with methods known in the art, and the IL-22BP expression levels of (CD4−) T-cells, in particular CD4+CD3+CD11− T-cells, are determined and compared.

In the following, the invention is described in more detail by way of examples and the attached figures for illustration purposes only.

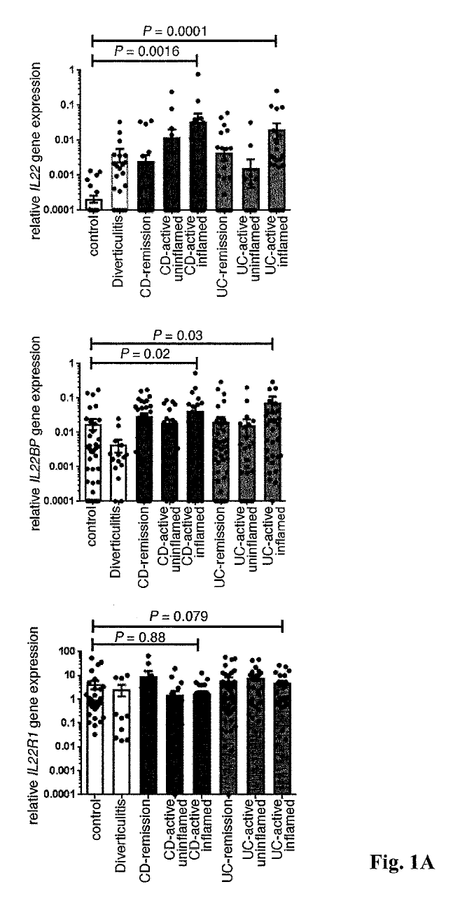

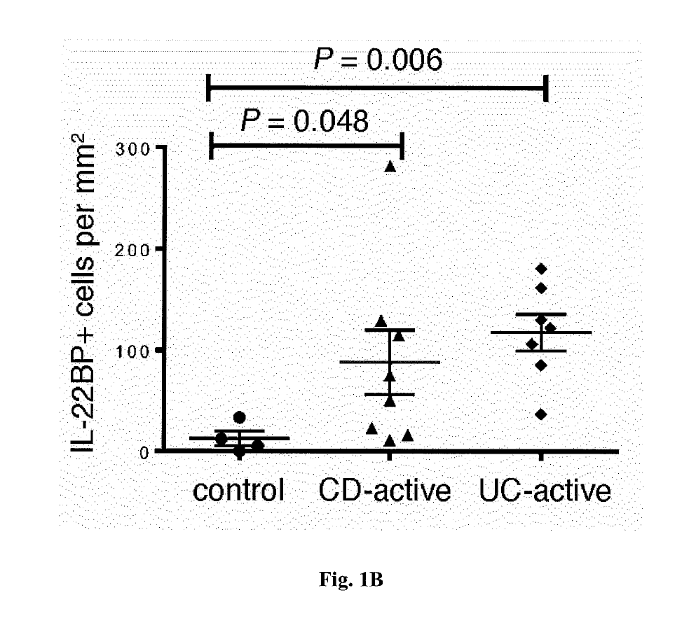

FIG. 1. Increased IL-22 and IL-22BP expression in IBD. (A) Relative IL22BP, IL22, and IL22R1 mRNA expression, as measured by reverse transcriptase polymerase chain reaction (RT-PCR) of intestinal specimens from patients with Crohn's disease (CD), ulcerative colitis (UC), and diverticulitis, as well as from healthy controls (number of patients: UC in remission, n=13; active UC, n=18; CD in remission, n=12; active CD, n=21; diverticulitis, n=5; controls, n=12). Filled circles represent one sample, bars represent means, and error bars show SEM. (B) quantitative assessment of IL-22BP immunohistochemical staining of colonic biopsy specimens (each symbol represents one patient; horizontal lines indicate means±SEM).

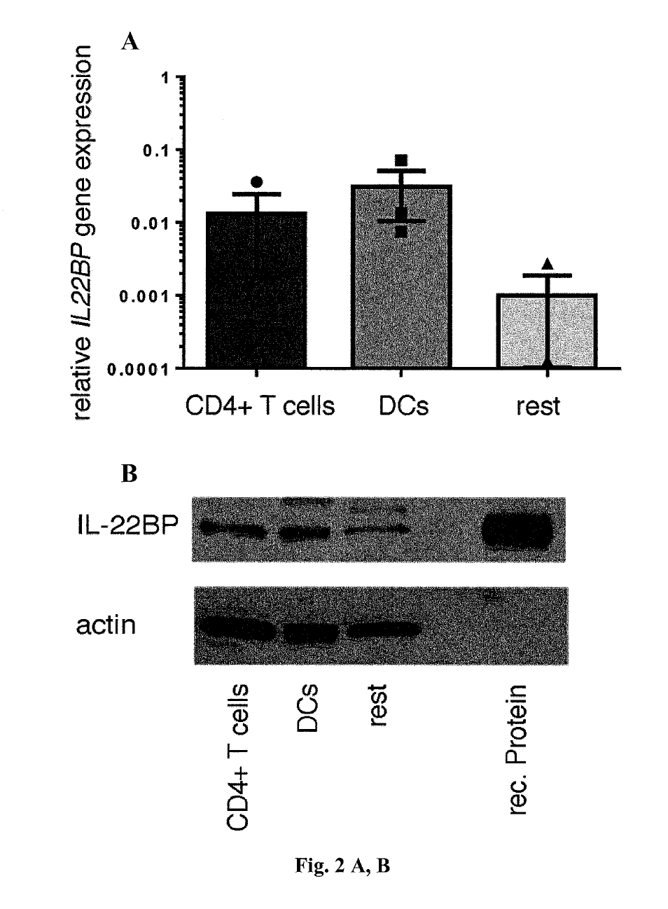

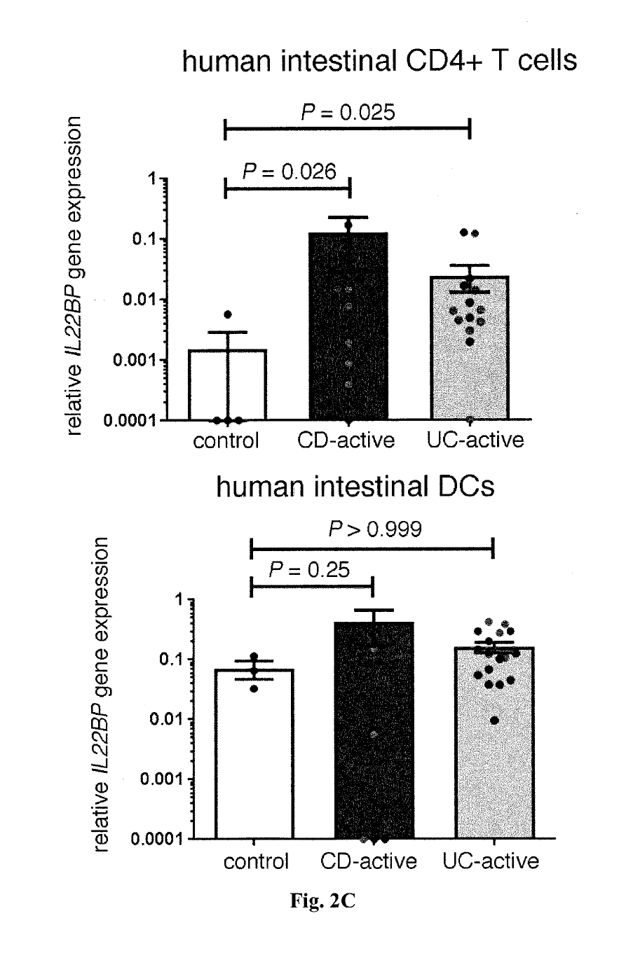

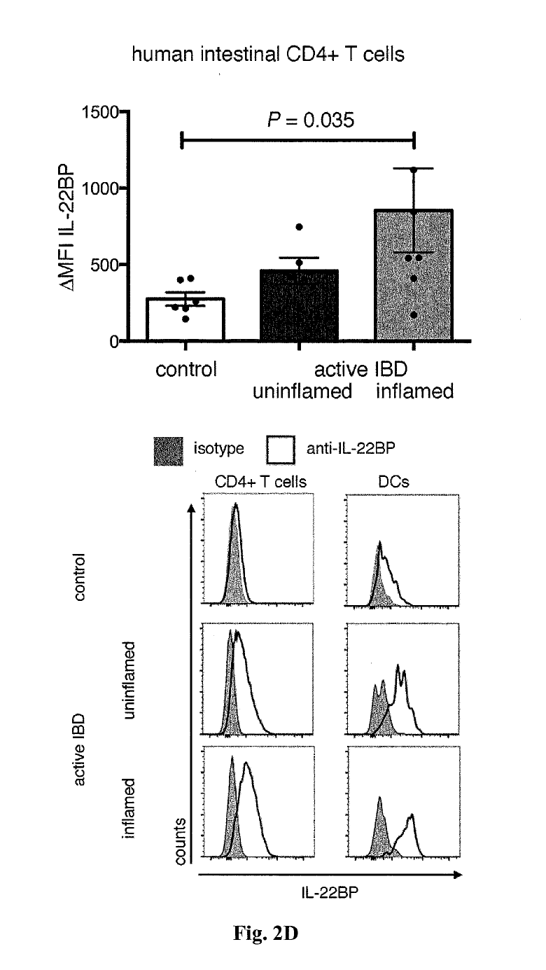

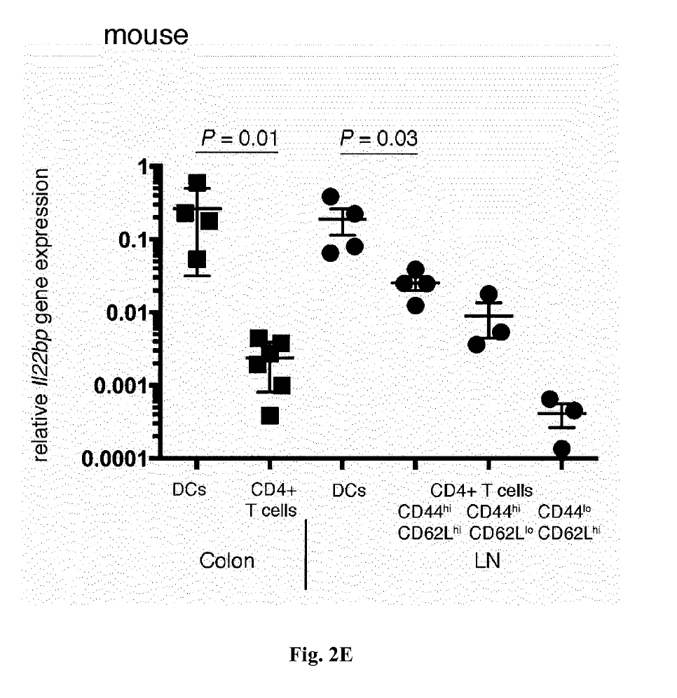

FIG. 2. CD4+ Tcells express IL-22BP in IBD. In (A) and (B), cells were isolated from the ileum of patients undergoing laparoscopic gastric bypass surgery because of morbid obesity. (A) Relative IL22BP mRNA expression, as measured by RT-PCR of sorted CD45+CD4+CD3+CD11c− T cells, CD45+CD11c+MHC-II+CD3− DCs, and CD45+CD4−CD3−CD11c− cells (rest). Bars represent means, error bars show SEM (n=3 patients). (B) A representative example of Western blot analysis of IL-22BP from sorted CD4+CD3+CD11c− T cells and CD45+CD4−CD3−CD11c− cells (rest). In vitro differentiated monocyte-derived DCs and recombinant (rec.) IL-22BP were used as positive controls. Results are representative of at least three independent experiments. (C) Relative IL22BP mRNA expression, as measured by RT-PCR of sorted CD4+CD3+CD11c− T cells and CD11c+MHC-II+CD3− DCs isolated from intestinal biopsy specimens from active CD, active UC, or healthy control patients. When possible, biopsies were taken from inflamed (grey circles) and uninflamed (black circles) regions of the intestine. Bars represent means, error bars show SEM (number of patients: control, n=4; CD, n=7; UC, n=11). (D) Analysis of IL-22BP expression by flow cytometry. Bars represent means, error bars show SEM, and each circle represents one sample (control, n=6; IBD unin-flamed regions, n=5; IBD inflamed regions, n=7) (number of patients: control, n=6; UC, n=4; CD, n=3). *One outlier not depicted. (E) Relative Il22bp mRNA expression, as measured by RT-PCR of sorted CD4+ T cells and DCs from murine colon and lymph node (LN). Horizontal lines indicate means±SEM; each symbol represents one experiment using pooled samples from three to six mice per group.

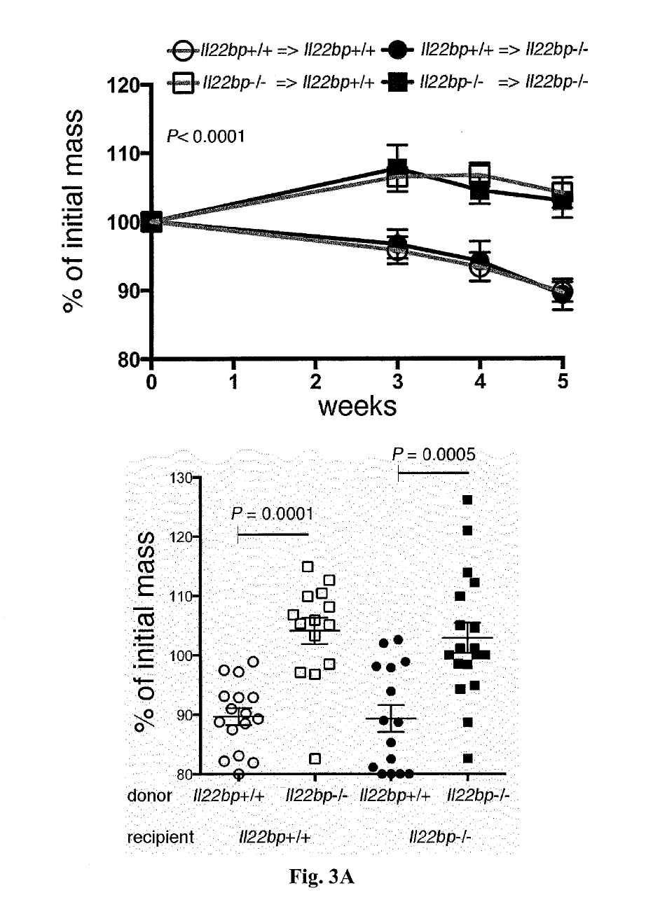

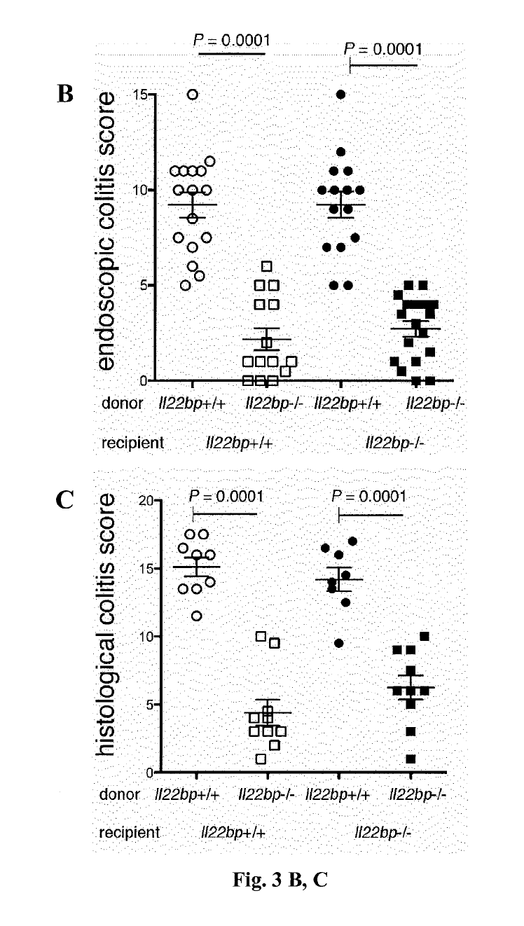

FIG. 3: A pathogenic role of CD4+ T cell-derived IL-22BP in a murine colitis model. CD4+CD25−CD45RBhigh cells were isolated from the spleen and lymph nodes of Il22bp+/+ and Il22bp−/− mice and transferred into Ragt−/− and Ragt−/−Il22bp−/− recipients. Disease development was assessed by (A) weight loss, (B) endoscopic, and (C) histological findings 5 weeks after transfer. Each symbol represents one mouse. Horizontal lines indicate means±SEM. Results are representative of four independent experiments.

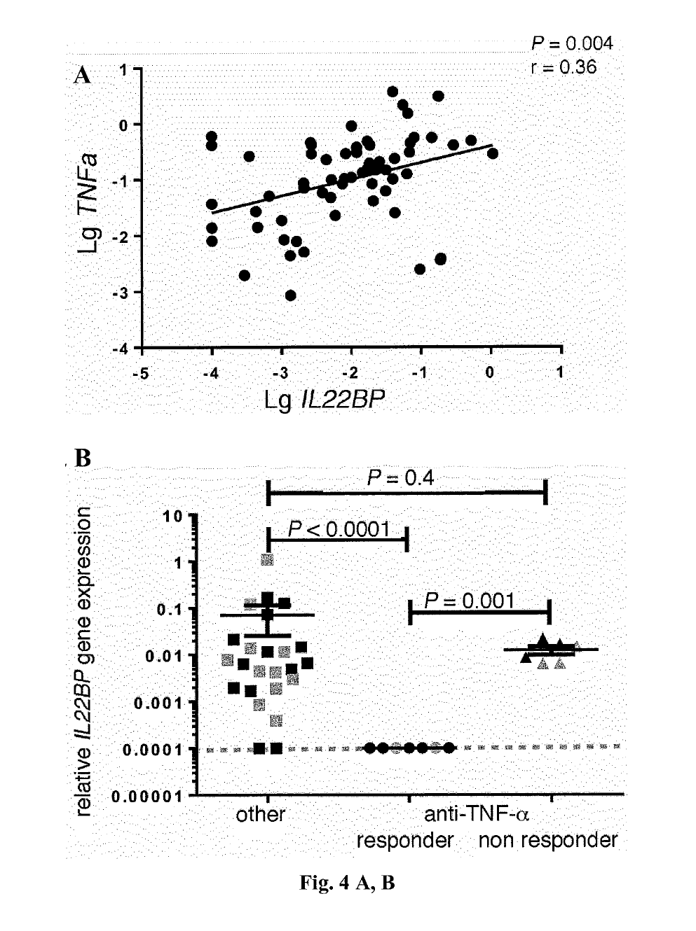

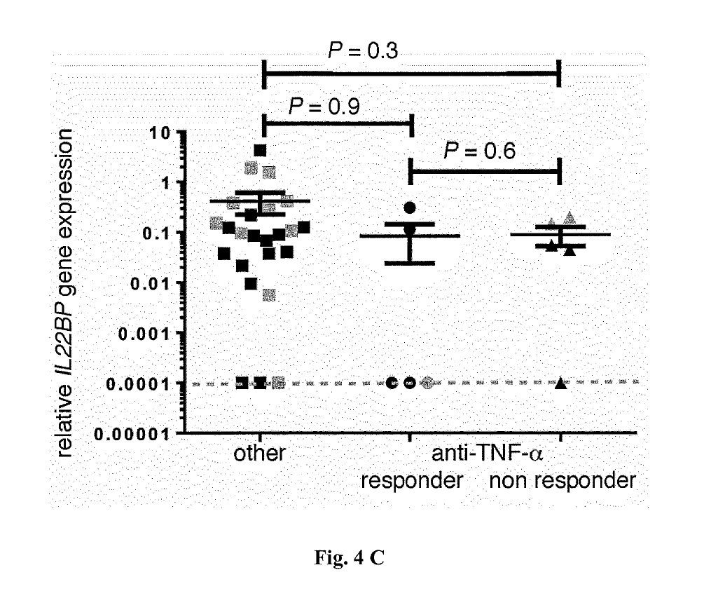

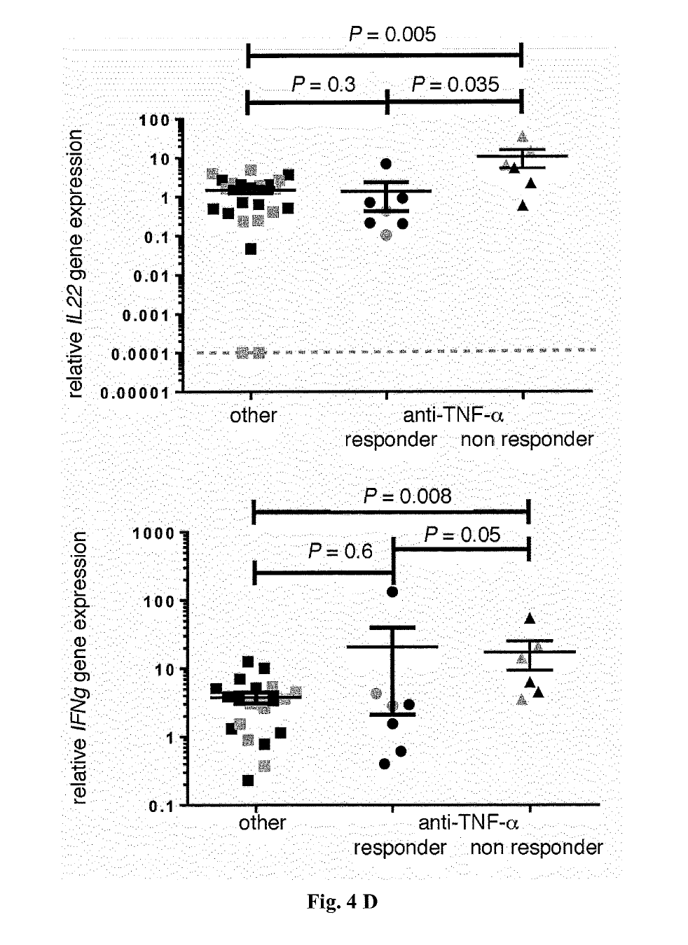

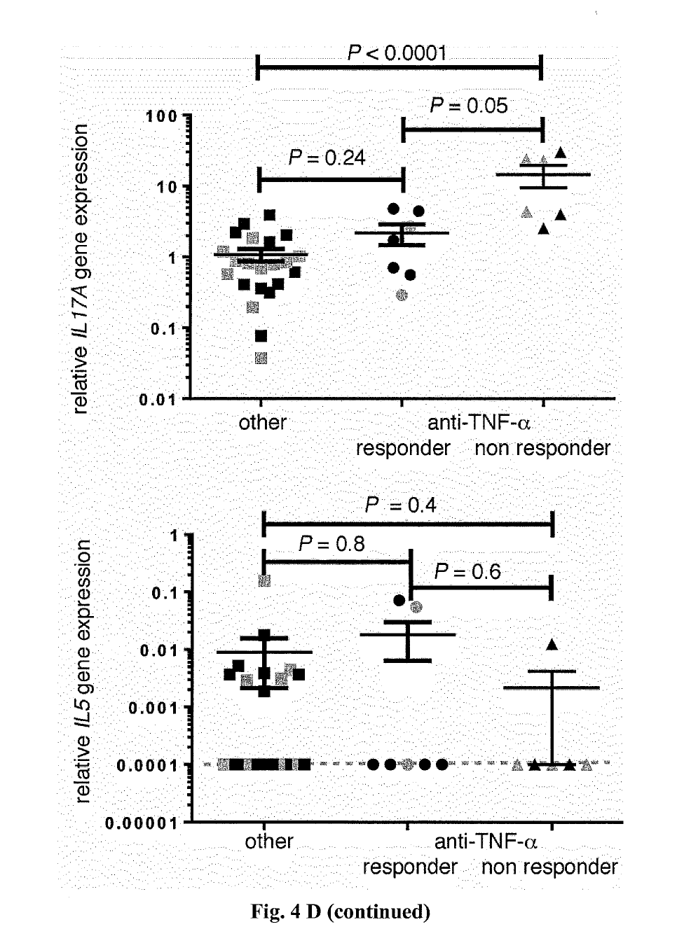

FIG. 4. Anti-TNF-a therapy correlates with reduced IL-22BP expression by CD4+ T cells in IBD (UC and CD) patients. (A) Correlation between TNFα and IL22BP mRNA expression, as measured by RT-PCR (P=0.004; r=0.36) of specimens from the intestines of patients with active IBD. In (B) to (D), CD4+CD3+CD11c− T cells and CD11c+MHC-II+CD3− DCs were isolated from intestinal biopsy specimens from patients with IBD (CD and UC). When possible, biopsies were taken from inflamed (grey circles) and uninflamed (black circles) regions. (B and C) Relative IL22BP mRNA expression, as measured by RT-PCR of (B) CD4+ T cells and (C) DCs isolated from intestinal biopsy specimens from IBD patients being treated with anti-TNF-a therapy or other therapies. (D) Relative IL22, IL17A, IFNg, and IL5 mRNA expression, as measured by RT-PCR of sorted CD4+T cells from IBD patients being treated with anti-TNF-α therapy or other therapies. Horizontal lines indicate means±SEM; dotted lines indicate the detection limit (number of patients: other therapies, n=16; anti-TNF-α responder, n=5; anti-TNF-α nonresponder, n=5).

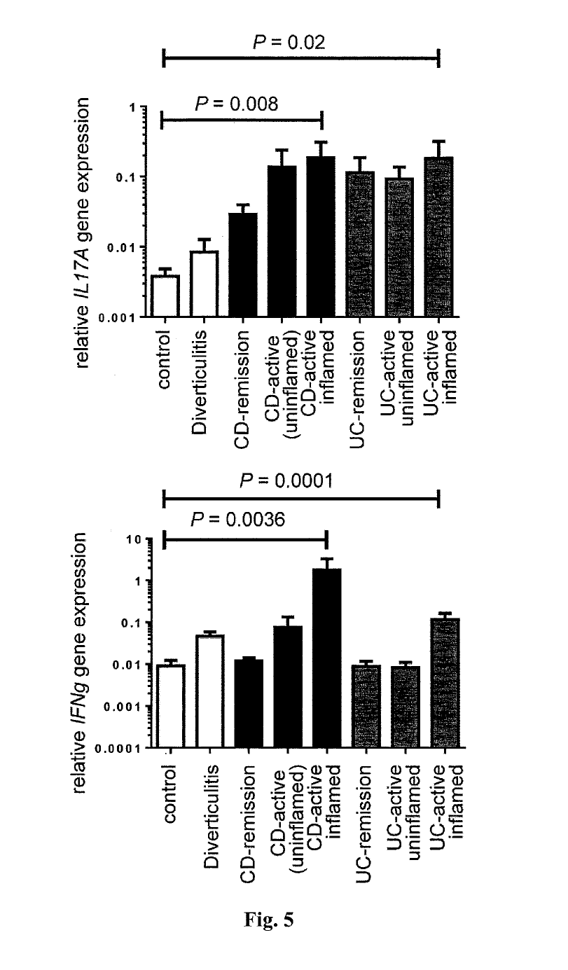

FIG. 5. Increased IL17A and IFNg expression in IBD. Relative IL17A and IFNg expression from intestinal specimen from patients with Crohn's disease (CD), ulcerative colitis (UC), diverticulitis and healthy controls (patient number: UC-remission=13, UC-active=18, CD-remission=12, CD-active=21, Diverticulitis=5, controls=12; Bars represent mean, error bars show SEM).

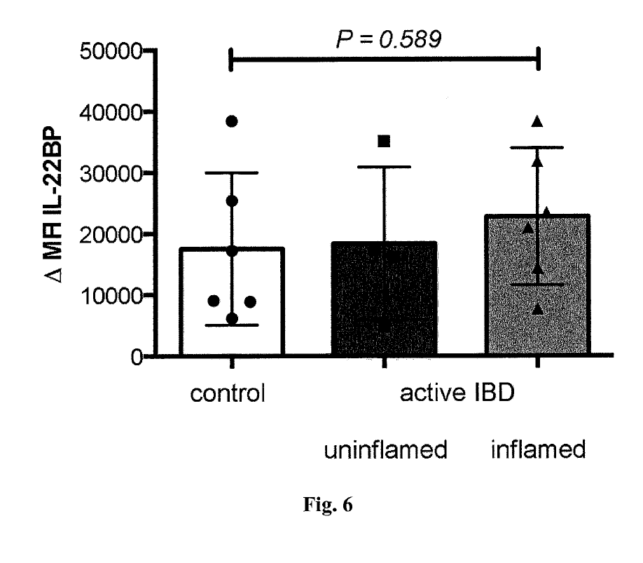

FIG. 6. IL-22BP expression by eosinophils. Cells were isolated from intestinal biopsy specimen of colon and terminal ileum of patients with IBD (CD or UC) or healthy controls. Analysis of IL-22BP expression in eosinophils using flow cytometry. Bars represent mean, error bars show SEM; each dot represents one sample; control=6, IBD uninflamed regions=5, IBD inflamed regions=7; patient number: control=6, UC=4, CD=3.

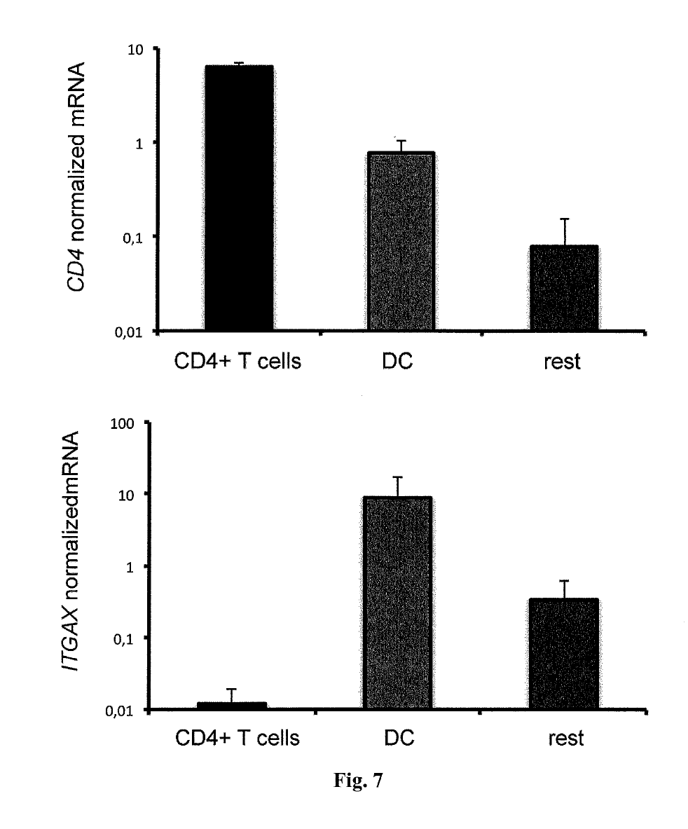

FIG. 7. CD4+ T cells are not contaminated by DCs. Relative CD4 and ITGAX (CD11c) expression from indicated cell fractions isolated from the small intestine. Bars represent mean, error bars show SEM. Data are cumulative from three independent experiments.

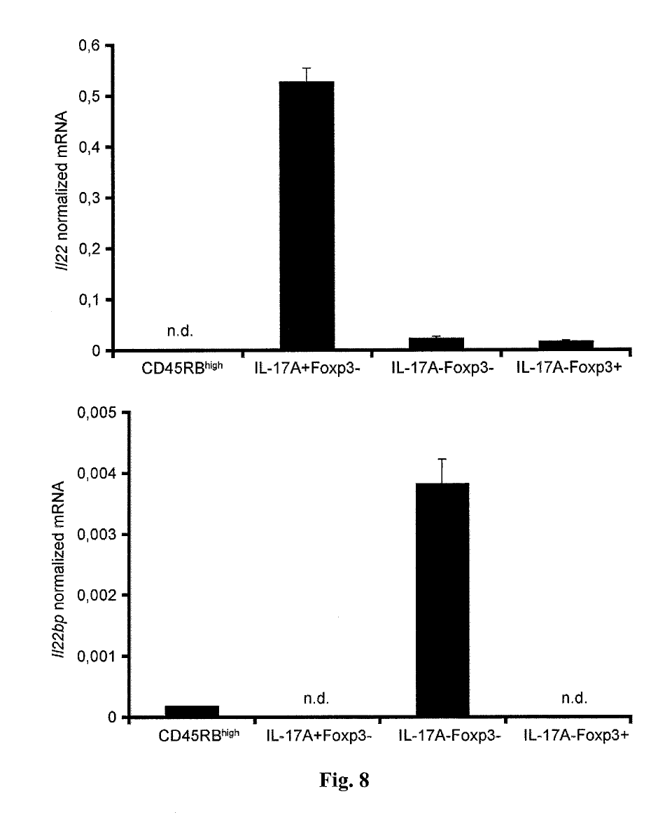

FIG. 8. Il22bp is expressed by IL-17A-Foxp3− T cells during Colitis. CD4+Foxp3− CD45RBhigh cells were isolated from spleen and lymph nodes of IL-17A eGFP×Foxp3 mRFP reporter mice and transferred into Rag1−/− recipients. 5 weeks after the transfer indicated CD4+ T-cell subsets were isolated from the intestine and Il22 and Il22bp expression analyzed using RT-PCR. Bars represent mean, error bars show SEM. Data are cumulative from two independent experiments. Cells were pooled from at least 4 animals in each experiment.

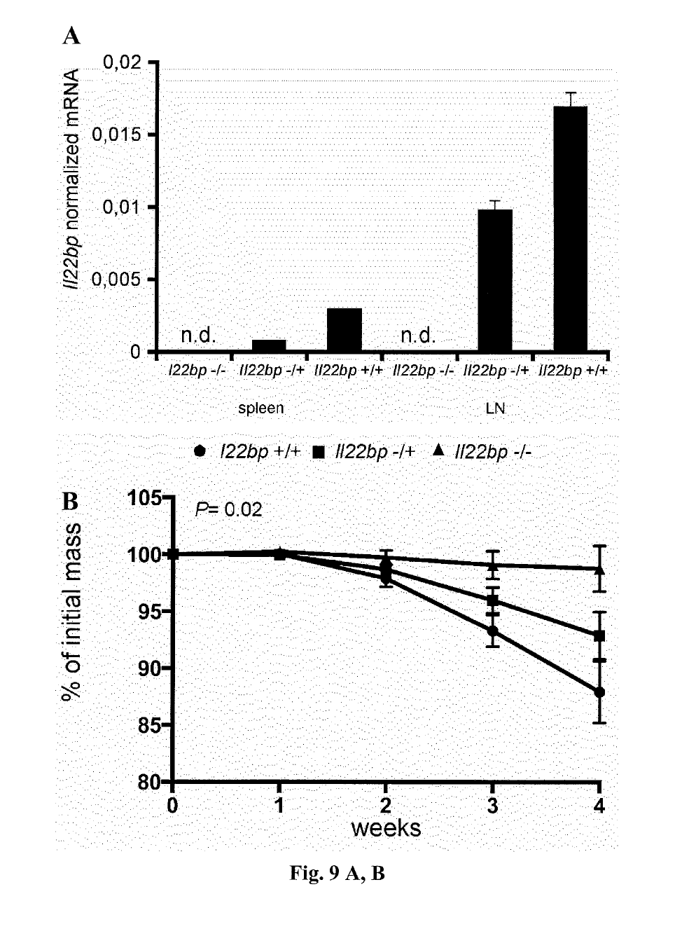

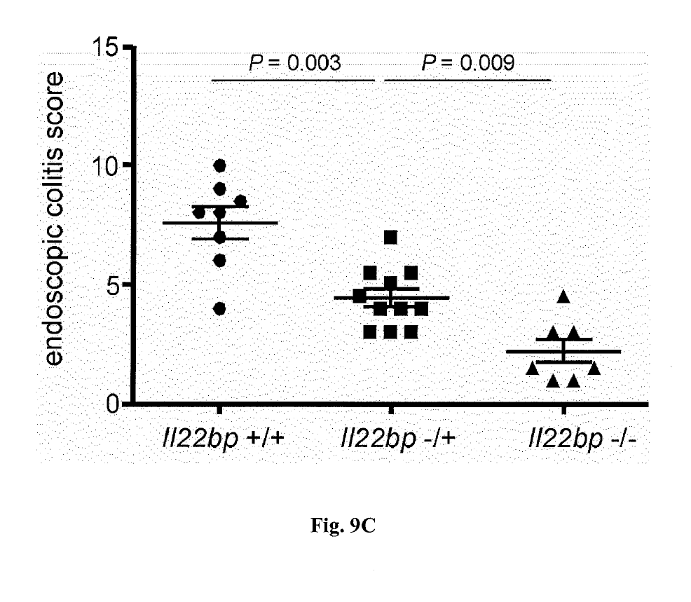

FIG. 9. A) Il22bp+/− T cells cause an intermediate colitis severity compared to Il22bp+/+ and Il22bp−/− T cells. Il22bp expression was analyzed in spleen and lymph nodes (LN) from Il22bp+/+, Il22bp−/+ and Il22bp−/− mice. Bars represent mean, error bars show SEM. B+C) CD4+CD25−CD45RBhigh cells were isolated from spleen and LN of Il22bp+/+, Il22bp−/− or Il22bp−/+ mice and transferred into Rag1−/− recipients. Colitis severity was assessed by weight loss (B) and endoscopy (C). Horizontal lines indicate mean +/−SEM. Each dot/square represents one mouse. Results are representative of two independent experiments.

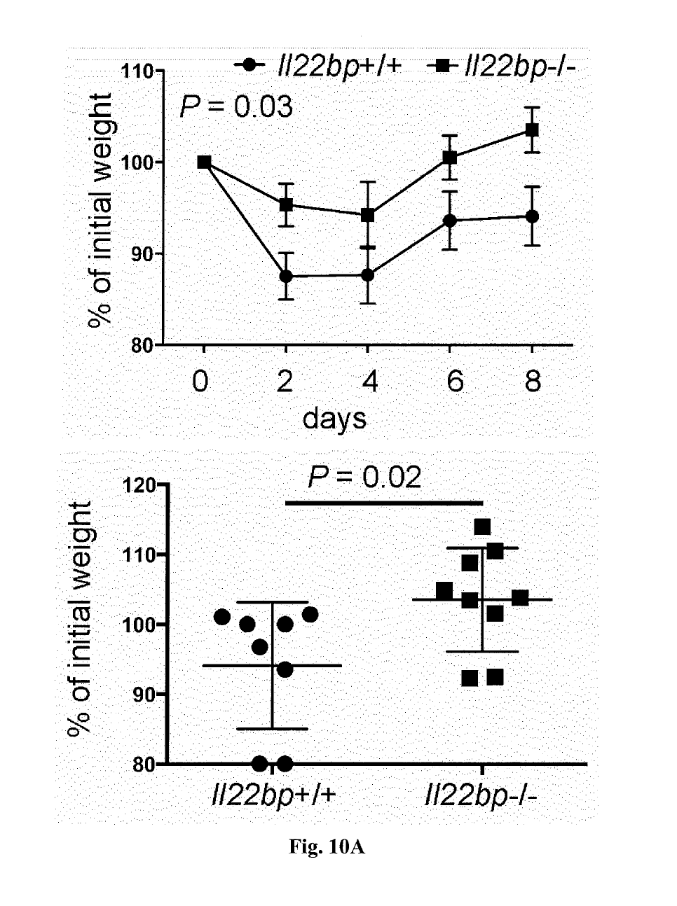

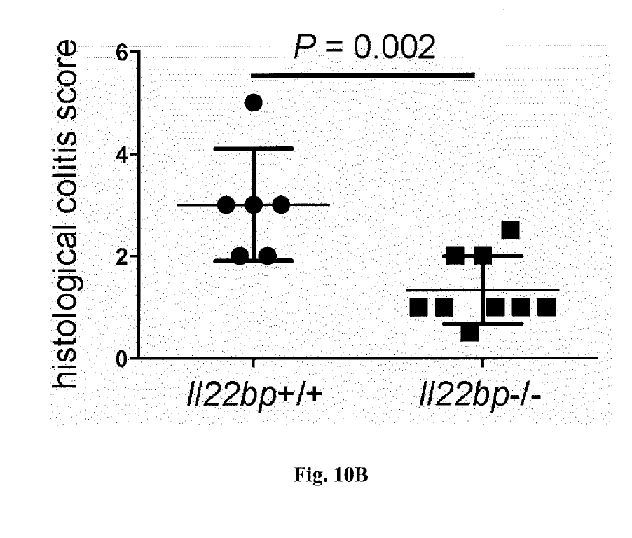

FIG. 10. A pathogenic role for T-cell derived IL-22BP during Citrobacter rodentium infection. CD4+CD3+ T cells were isolated from spleen and lymph nodes of Il22bp+/+ and Il22bp−/− mice and transferred into Rag1−/− recipients. 5 weeks after the transfer mice were infected with Citrobacter rodentium. Disease development was assessed by weight loss (A) and histological findings (B) at day 8 upon infection. Average score for edema, inflammation, hyperplasia, crypt loss, and ulceration is shown in B. Each dot represents one mouse. Horizontal lines indicate mean +/−SEM. Results are cumulative of 2 independent experiments.

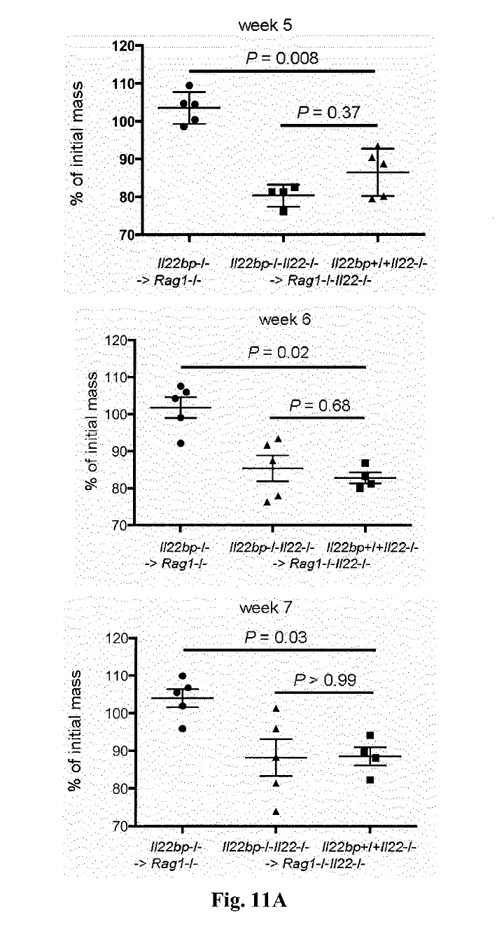

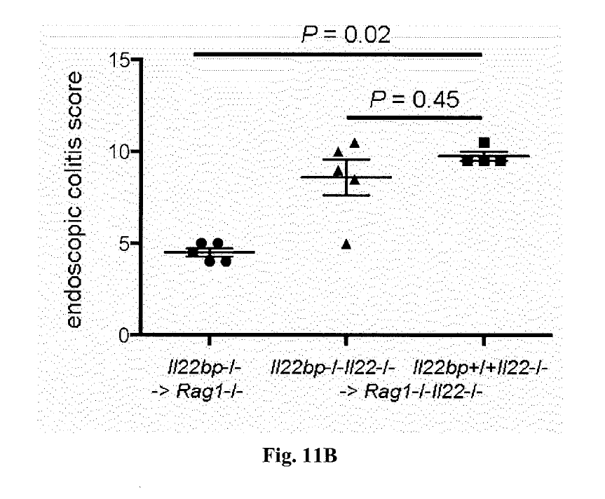

FIG. 11. The effect of IL-22BP is dependent on the presence of IL-22. Il22bp−/−, Il22−/− or Il22bp−/−Il22−/− CD4+CD25−CD45RBhigh were transferred into Rag1−/− or Rag1−/−Il22−/− mice. Weight loss (A) and endoscopic colitis score (B) are shown. Horizontal lines indicate mean +/−SEM. Each dot represents one mouse. Results are representative of two independent experiments.

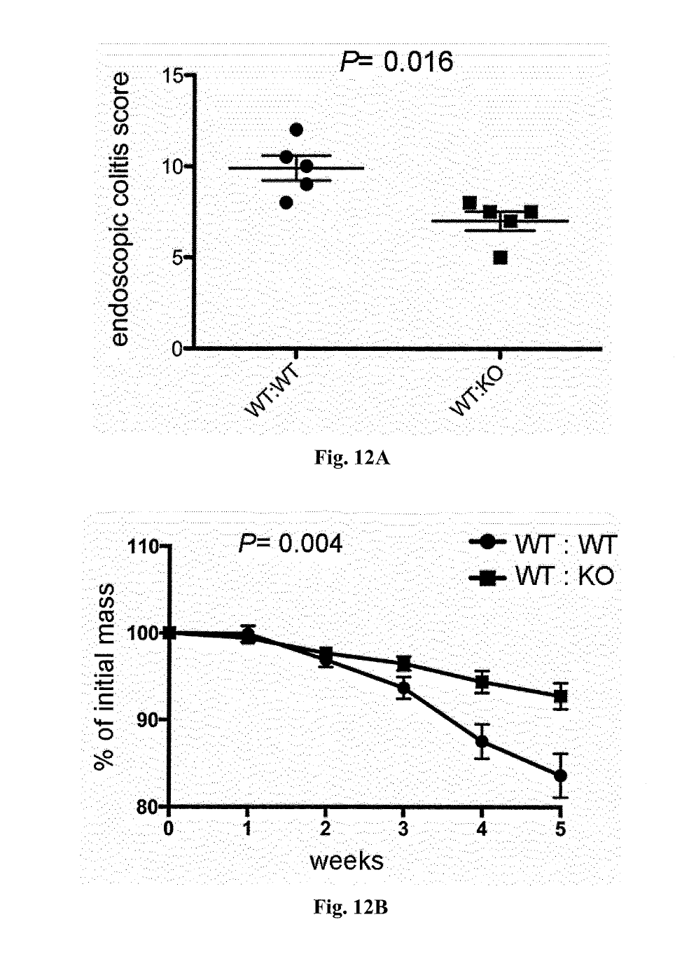

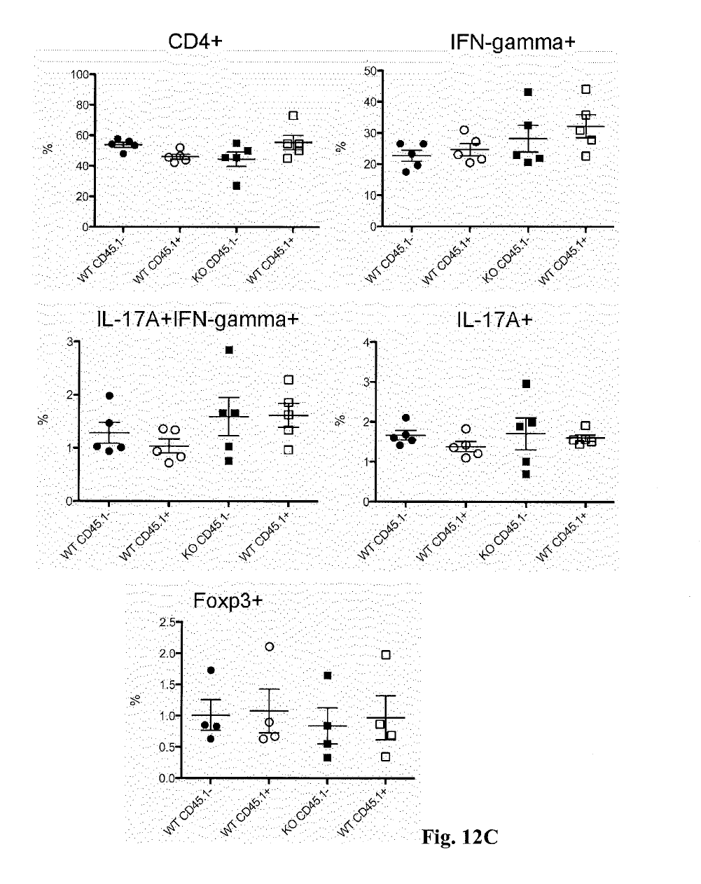

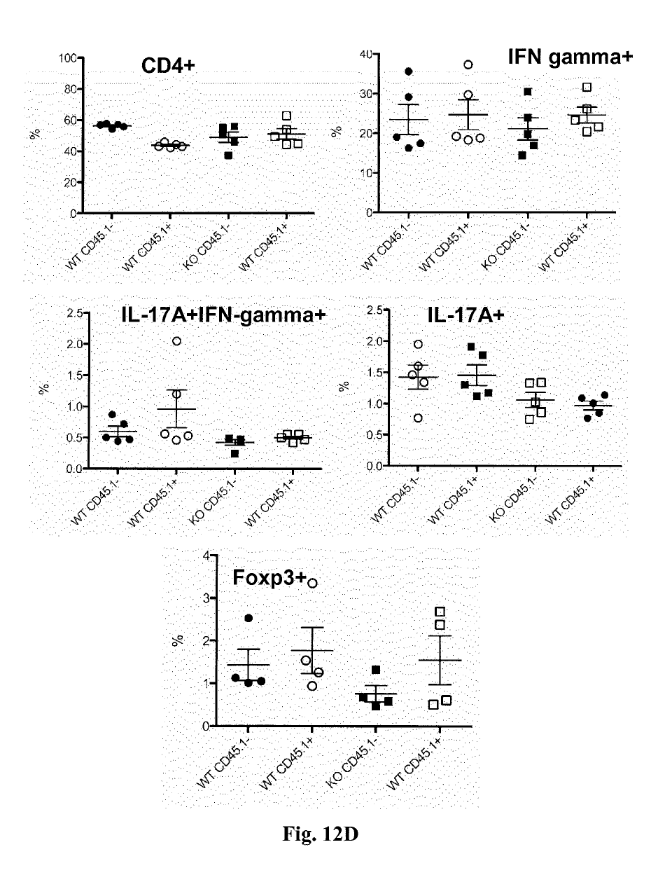

FIG. 12. Il22bp−/− T cells do not have a cell intrinsic defect. Wild type or Il22bp−/− CD4+CD25−CD45RBhigh were co-transferred with congenic wild type cells into Rag1−/− mice. Endoscopic colitis score (A) and weight loss (B) 5 weeks upon transfer. Cells were isolated from colon (C) and spleen (D) and analyzed by flow cytometry. Horizontal lines indicate mean +/−SEM. Each dot represents one mouse. Results are representative of two independent experiments.

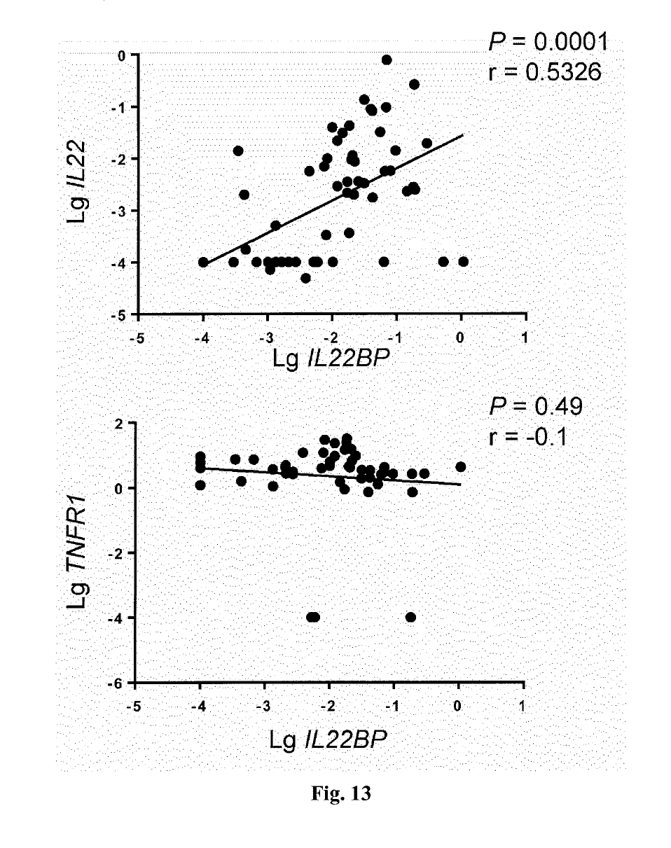

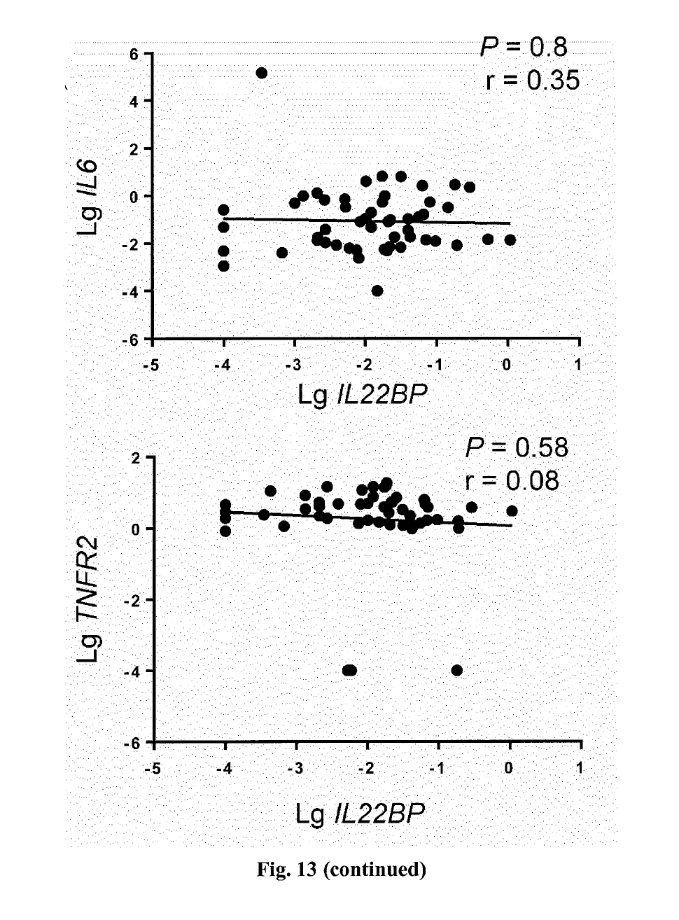

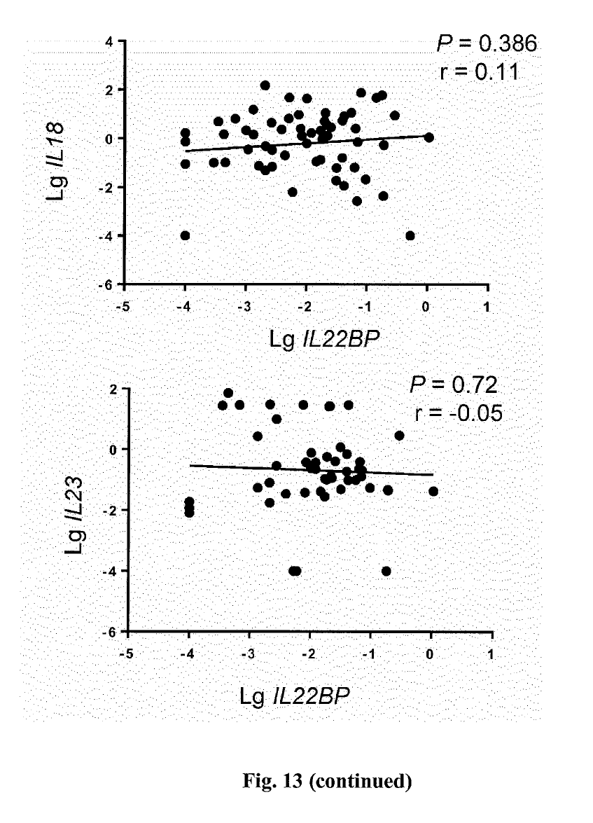

FIG. 13. Correlative analysis of indicated genes and IL22BP from intestinal biopsies of patients with active IBD. Each dot represents one sample.

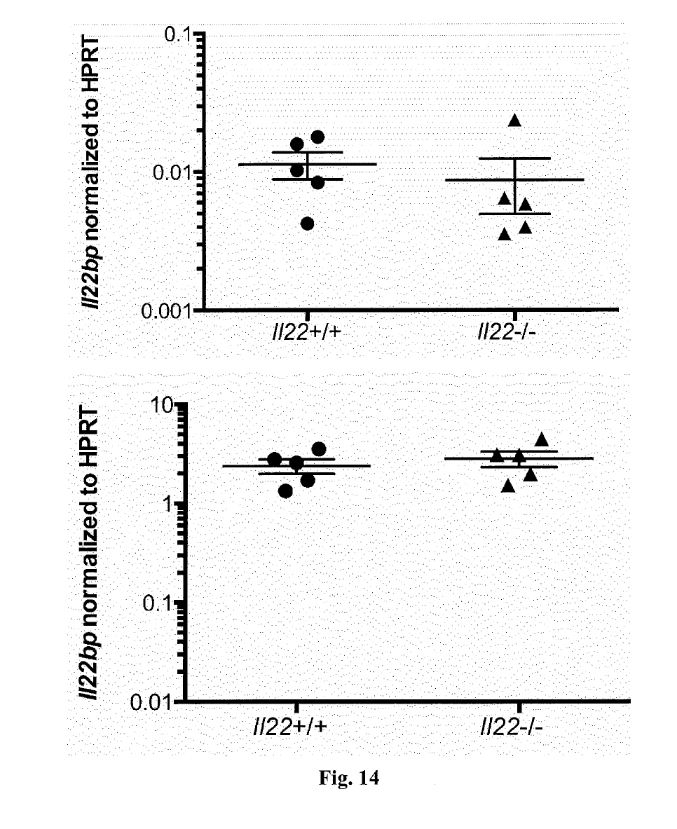

FIG. 14. Il22bp expression is not altered in Il22−/− mice. RNA was isolated from the colon (top) and lymph nodes (bottom) of wild type and Il22−/− mice. Il22bp expression was measured using RT-PCR. Horizontal lines represent mean +/−SEM. Each dot represents one mouse. Results are representative of two independent experiments.

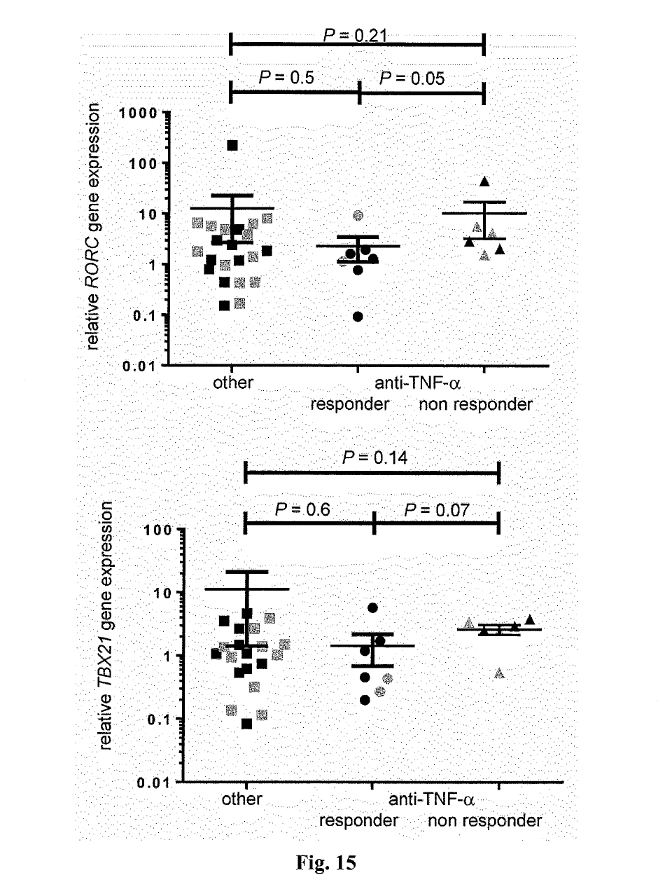

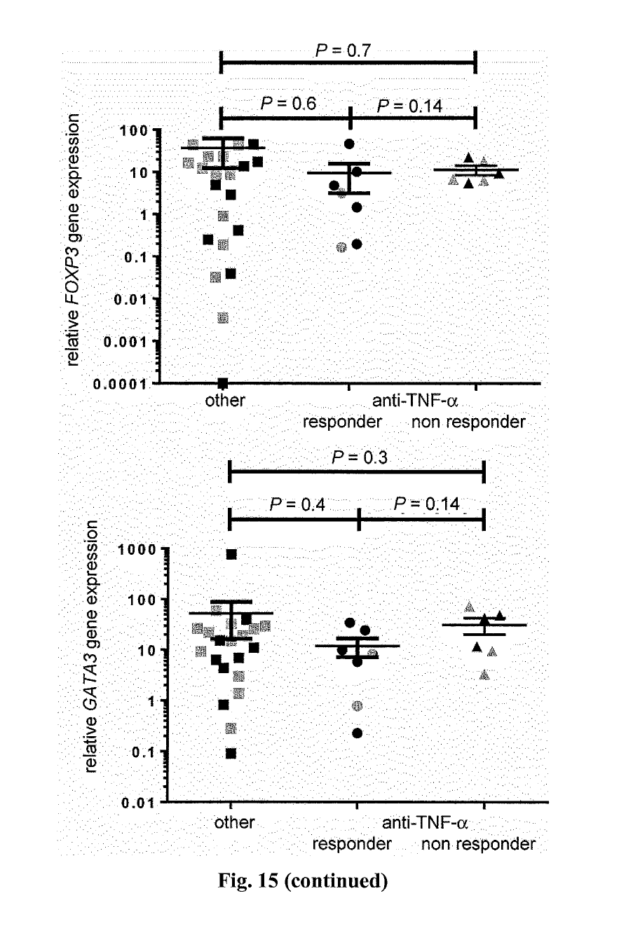

FIG. 15. RORC, FOXP3, TBX21, and GATA3 expression by CD4+ T cells is not influenced by anti-TNF-α therapy. Relative RORC, FOXP3, TBX21, and GATA3 gene expression of CD4+ T cells isolated from intestinal biopsy specimen from IBD patients with anti-TNF-alpha therapy compared to other therapies is shown. When possible biopsies were collected from inflamed (grey) and uninflamed (black) regions (patient number; other medication n=16, responder n=5, non responder n=5). Each dot represents one sample. Horizontal lines indicate mean+/−SEM.

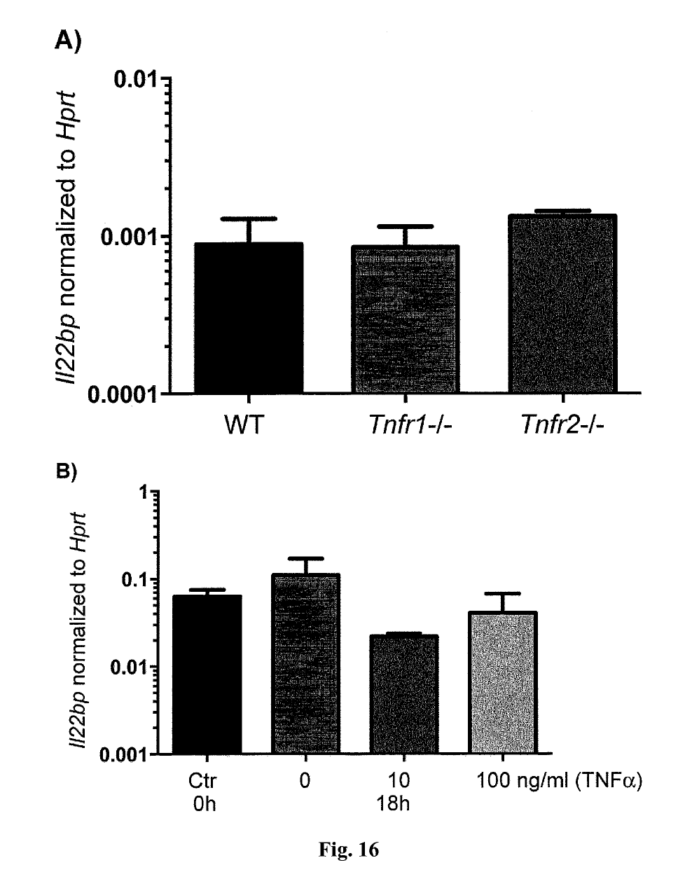

FIG. 16. Role of TNF-α for IL-22BP expression by T cells. A) CD4+CD25−CD45RBhigh cells were isolated from spleen and LN of WT, Tnfr1−/− and Tnfr2−/− mice and transferred into Rag1−/− recipients. 3 weeks after the transfer CD4+ T-cell subsets were isolated from the intestine and Il22bp expression analyzed using RT-PCR. Samples were pooled from at least three mice per group. Mean +/−SEM are shown (WT: n=3, Tnfr1−/−: n=4, Tnfr2−/−: n=3). Results are cumulative from two independent experiments. B) Memory T cells (Ctr: freshly isolated) were sorted from WT mice and cultured in the presence of TNFα at indicated concentrations for 18 hours. Il22bp expression was analyzed using RT-PCR. Mean +/−SEM from three independent experiments is shown.

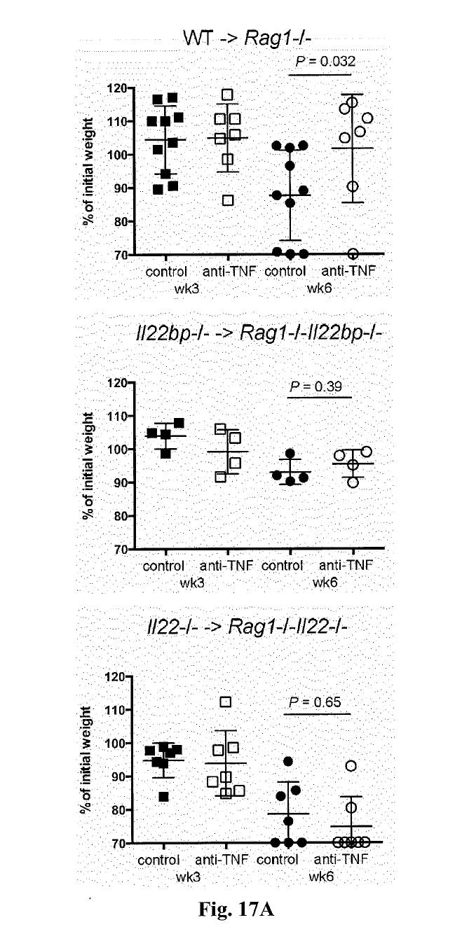

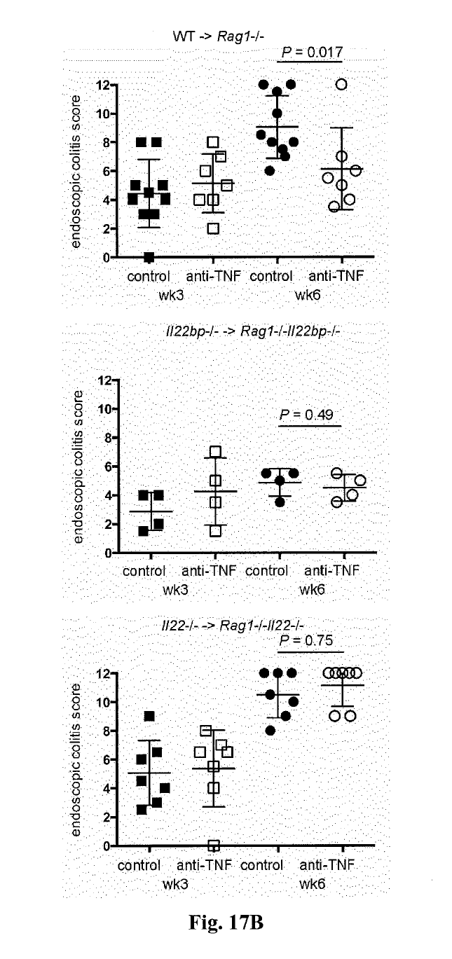

FIG. 17. The efficacy of anti-TNF-α therapy is dependent on the presence of IL-22 and IL-22BP. CD4+CD25−CD45RBhigh cells were isolated from spleen and lymph nodes of wild type, Il22−/− or Il22bp−/− mice and transferred into Rag1−/−, Rag1−/−Il22−/− or Rag1−/−Il22bp−/− recipients as indicated. Three weeks upon transfer mice were treated weekly with anti-TNF-alpha or isotype control (5 mg/kg body weight). Disease development was assessed by weight loss (A) and endoscopic findings (B). Each dot/ square represents one mouse. Horizontal lines indicate mean +/−SEM. Results are cumulative of 2 independent experiments.

### EXAMPLES

### Material and Methods

### Animals

Il22−/− and Il22bp−/− mice are described elsewhere [10, 8. Rag1−/−, Tnfr1−/−, and Tnfr2−/− mice were obtained from the Jackson Laboratory. Age- and sex-matched knock out mice and wild type littermates (in case of Il22−/−, Il22bp−/−) or co-housed in house breed C57/BL6 wild-type mice (in case of Tnfr1−/− and Tnfr2−/− mice) between 8 to 14 weeks of age were used for all experiments. All animals were cared for in accordance with the Institutional Animal Care and Use Committee of Yale University or the institutional review board ‘Behörde für Soziales, Familie, Gesundheit and Verbraucherschutz’ (Hamburg, Germany).

### Transfer-Colitis

Lymphocytes from spleen and lymph nodes were collected from 8 to 12 weeks old wild-type mice and indicated knock out strains. CD4+ T cells were enriched using MACS (Miltenyi Biotec GmbH) following the manufacturer's instructions and further sorted to collect CD25−CD45RBhi cells using a FACS Aria II. 4×105 CD4+CD25−CD45RBhi cells were injected intraperitoneally into Rag1−/−, Il22bp−/−Rag1−/− or Il22−/−Rag1−/− mice as indicated. Colitis development was measured by changes in weight, endoscopic and histological findings. At indicated time points the mice were sacrificed and lymph nodes and colon were isolated for further analysis.

Infection with Citrobacter rodentium 6 weeks old Rag1−/− mice were repopulated with 4×10⁶ CD4+ T cells from either Il22bp−/− or wild-type mice. 5 weeks upon transfer mice were orally gavaged with C. rodentium (109 CFU/200 μl). On day 8 post-infection the mice were sacrificed and the colon and caecum was isolated for analysis.

### Endoscopic and Histopathological Procedures

Colonoscopy was performed in a blinded fashion for colitis scoring using the Coloview system (Karl Storz, Germany) as previously described [27]. In brief: Colitis scoring was based on granularity of mucosal surface, stool consistency, vascular pattern, translucency of the colon and the visibility of fibrin (0-3 points for each). For histological colitis scoring colons were evaluated and were assigned scores by investigators blinded to experimental manipulation. Each section was evaluated by a semiquantitative criterion based method (score 0-5 for edema, inflammation, hyperplasia, crypt loss, and ulceration) essentially as described before [28].

### Human Samples

Small intestinal samples were taken from the ileum part of the small intestine from patients undergoing laparoscopic gastric bypass surgery due to morbid obesity. Paired endoscopic biopsy specimens were obtained from the colon (ascending colon, transverse colon, descending colon, sigma/rectum) and terminal ileum from patients with IBD or suspicion of intestinal disease. An additional paired biopsy was taken from the largest foci of macroscopic inflammation. One of the paired biopsies was used for histological assessments and the other one for RNA extraction or cell isolation. Colon biopsies of diverticulitis were obtained from patients undergoing surgical resection due to recurrent diverticulitis. Disease severity was based on Mayo score for UC (remission: 0-2, mild: 3-5, moderate: 6-10, severe 11-12 points) and Harvey-Bradshaw index for CD (remission: 0-4, mild: 5-7, moderate: 8-16, severe >16 points). For comparison of IBD patients we assigned scores for disease severity (remission: 0, mild: 1, moderate: 2, severe 3 points). Human studies were approved by the local ethical committee (Ethik-Kommission der Ärztekammer Hamburg PV4444).

Isolation of Hematopoietic Cells from Murine and Human Intestine

Hematopoietic cells were isolated from freshly obtained human colon biopsies, human small intestine or murine colon. From human small intestine samples the mucosa was isolated. After removal of the Peyer's patches and the adventitial fat the murine colon was cut longitudinally. Prepared samples were washed with PBS. For isolation of intraepithelial lymphocytes (IEL) the intestinal tissue was incubated in HBSS containing 1 mM dithioerythritol (DTE) followed by a dissociation step using 1.3 mM EDTA for 20 min at 37° C. respectively. To isolate lamina propria lymphocytes (LPL) the tissue was further cut in small pieces and minced with a scalpel. The remaining tissue was incubated for 45 min at 37° C. on a shaking incubator in HBSS (with Ca²⁺ and Mg²⁺) with Collagenase (1 mg/ml) and DNase I (10 U/ml) and supernatant was collected. Leukocytes were further enriched by Percoll gradient centrifugation (GE Healthcare). If not stated otherwise, IEL and LPL were collected and pooled.

### Histopathology Procedures

Immunohistochemistry was performed on 5 μm formalin-fixed and paraffin-embedded sections of human colonic biopsies. Slides were deparaffinized and exposed to heat-induced antigen retrieval for 5 minutes in an autoclave at 121° C. in pH 7.8 wash buffer (Dako, Glostrup, Denmark) and primary antibody specific for IL-22BP (dilution 1:450; R&D Systems) was applied.

Bound antibody was then visualized using the EnVision Kit (Dako). All sections were counter-stained with hematoxylin. IL-22BP+ cells were counted in a blinded fashion from at least 4 areas of a given histological section and divided by the total tissue surface obtained from the 4 areas. For indicated analysis histological slides were stained with Haematoxylin and Eosin.

### RNA Analysis

Total RNA was extracted from tissue and cells of the small intestine, colon and lymph nodes using Trizol® Reagent (Invitrogen). The High capacity cDNA synthesis Kit (Applied Biosystems) was used for synthesis of cDNA. Primers and probes were purchased from Applied Biosystems. Human primers and probes including reference: IL-22 (Hs01574154_m1), IL-22RA1 (Hs00222035_m1), IL-22RA2 (Hs00364814_m1), IL-17A (Hs00174383_m1), IL-23 (Hs00900828_g1), IL-18 (Hs01038788_m1), IFN-γ (Hs00989291_m1), TNF (Hs01113624_g1), and HPRT1 (Hs02800695_m1), IL-6 (HS00189606_m1), IL-5 (Hs01548712_g1), TNFR1 (Hs01042313_m1) and TNFR2 (Hs00961749_m1), CD4 (Hs01058407_m1), CD11c (Hs00174217_m1), FOXP3 (Hs01085834_m1), GATA-3 (Hs00231122_m1), TBX21 (Hs00203436_m1), RORC (Hs01076122_m1).

Mouse primers and probes including reference: HPRT (Mm01545399_m1), IL-22 (Mm00444241_m1), IL-22RA2 (Mm01192969_m1).

Real-time PCR was performed using the Kapa Probe Fast qPCR Master Mix (Kapa Biosystems) on the StepOne Plus system (Applied Biosystems). For both human and mouse, relative expression was normalized to HPRT and calculated using the 2-ΔΔCt method.

### Western Blot

Western blot analyses were undertaken as previously described [29]. Total protein extracts (50m) were separated by electrophoresis and transferred onto nitrocellulose membranes. Blots were incubated with IL-22BP antibody (MAB1087, R&D Systems) at 1:1000, polyclonal rabbit anti-mouse HRP (Dako) at 1:2000 and reprobed with mouse anti-actin (Santa Cruz). Recombinant IL-22BP was purchased from Sino Biological.

### Flow Cytometry

Human antibodies anti-CD45, anti-CD4, anti-CD3, anti-CD11c, anti- Siglec-8, and mouse antibodies anti-CD45.2, anti-CD45.1, anti-MHCII, anti-CD11c, anti-CD3, anti-CD4, anti-CD44, anti-CD62L, anti-CD25, anti-Foxp3, anti-IFNγ, anti- IL-17A, and anti-CD45RB were purchased from Biolegend. Anti-human CD3 and anti-human CD4 were purchased from BD Biosciences. Anti-human IL-22BP antibody (clone 87554) and IgG2B isotype control were purchased from R&D Systems. To identify dead cells, 7-AAD staining (Biolegend) was performed. For extracellular staining isolated hematopoietic cells of the small intestine, the colon and the lymph nodes were incubated for 20 min at 4° C. Cells were sorted on a FACS Aria II or acquired on a LSRII Fortessa flow cytometer (BD), respectively. Data were analyzed with FlowJo software (Treestar).

### CBA

For serum cytokine quantification LEGENDplexTM human T helper cell cytokine panel was used. Experimental procedures have been performed according to the manufacturer's instructions.

### Statistical Analysis

Statistical analysis was performed with GraphPad Prism® Software (GraphPad Software, San Diego, Calif., USA). For comparison of groups, the non-parametric two-sided Mann-Whitney test was used. Bonferroni correction was used to counteract the problem in case of multiple comparisons. For time-dependent weight-loss data a repeated-measures ANOVA to assess the significance of the main effects and an experimental group-time interaction was used. The significance level a was set to 0.05. The Pearson correlation was used for correlative analyses. The significance level was set to 0.05.

### Results

IL-22 and IL-22BP expression in intestinal biopsy specimens from patients with ulcerative colitis (UC) or Crohn's disease (CD) (table 1) who either were in remission (UC, n=13; CD, n=12) or had active disease based on clinical, endoscopic, and histological findings (UC, n=18; CD, n=21) was analyzed.

TABLE 1
Patient characteristics. Years are mean ± SD.

| | | UC- | UC- | CD- | CD- | |
|---|---|---|---|---|---|---|
| | Control | remission | active | remission | active | Diverticulitis |
| | (n = 12) | (n = 13) | (n = 18) | (n = 12) | (n = 21) | (n = 5) |
| Sex (F/M) | 9/3 | 6/7 | 8/10 | 8/6 | 9/18 | 1/4 |
| Age (years) | 43 ± 18.6 | 42 ± 13.0 | 44 ± 13.8 | 41 ± 13.7 | 40 ± 12.6 | 46 ± 11.3 |
| Involvement UC | | | | | | |
| left sided | | | 5 | | | |
| distal | | | 10 | | | |
| pancolitis | | | 3 | | | |
| Involvement CD | | | | | | |
| ileum | | | | | 7 | |
| colon | | | | | 8 | |
| ileum + colon | | | | | 6 | |

In patients with active IBD, we analyzed samples from both healthy (uninflamed) and diseased (inflamed) areas in the colon and terminal ileum, based on endoscopic and histological findings, which were further validated by analyzing expression of the genes for the inflammatory markers IL-22, IL-17A, and interferon-γ (IFN-γ) (FIG. 1A and FIG. 5). In line with previous reports [3, 13], we found increased IL22 and IL17A mRNA expression in the colon and terminal ileum of patients with active CD and UC, compared with that in healthy controls. IL22BP mRNA was not decreased, but was rather increased in the colon of these patients (FIG. 1A), suggesting that the activity of IL-22 might be impaired. We also confirmed this finding at the protein level by immunohistochemistry of intestinal biopsy specimens (FIG. 1 B). As a control, we analyzed the IL-22 and IL-22BP expression pattern in a non-IBD-related intestinal disease, colonic diverticulitis (n=5). Colonic diverticulosis is an acquired disease, developing as mucosal and submucosal herniation through the circular muscle layer at vulnerable weak points of the colonic wall. Subsequent inflammation of these diverticula is termed diverticulitis. We obtained tissue specimens from the inflamed area of the colon from patients with diverticulitis and, in line with the studies in mice [3, 10, 21], we observed increased IL22 and decreased IL22BP expression (FIG. 1A), suggesting that IL-22BP is not generally up-regulated during intestinal inflammation in humans. One possible explanation for the increased expression of IL-22BP in IBD relative to that in diverticulitis might be the chronic versus acute nature of the inflammatory response in these respective diseases.

In order to identify the cellular source of IL-22BP in the intestine, different cell populations from human intestine were sorted. In line with previous publications [10, 22, 9, 26], it was confirmed that dendritic cells (DCs) and eosinophils express high levels of IL-22BP (FIG. 2 A and B, and FIG. 6). Unexpectedly, high IL-22BP expression in CD4+CD3+CD11c− T cells in the small intestine (FIG. 2, A and B) was also observed. CD4 and CD11c (ITGAX) were measured as controls (FIG. 7). IL-22BP was also expressed in the colon by T cells, and it was further increased in T cells but not in DCs in the diseased area in patients with active IBD (FIG. 2 C and D, and tables 2 and 3).

TABLE 2
Patient characteristics. Years are mean ± SD.

| | | UC- | CD- |
|---|---|---|---|
| | Control | active | active |
| | (n = 4) | (n = 11) | (n = 7) |
| Sex (F/M) | 1/3 | 4/7 | 2/5 |
| Age (years) | 39 ± 18.2 | 47 ± 14.5 | 31 ± 7.9 |
| Involvement UC | | | |
| left sided | | 3 | |
| distal | | 5 | |
| pancolitis | | 3 | |
| Involvement CD | | | |
| ileum | | | 6 |
| colon | | | 0 |
| ileum + colon | | | 1 |

TABLE 3
Patient characteristics. Years are mean ± SD.

| | | UC- | CD- |
|---|---|---|---|
| | Control | active | active |
| | (n = 6) | (n = 4) | (n = 3) |
| Sex (F/M) | 2/4 | 2/2 | 3/0 |
| Age (years) | 49 ± 15.0 | 59 ± 16.2 | 31 ± 1.0 |
| Involvement UC | | | |
| left sided | | 1 | |
| distal | | 1 | |
| pancolitis | | 2 | |
| Involvement CD | | | |
| ileum | | | 0 |
| colon | | | 2 |
| ileum + colon | | | 1 |

Murine CD4+ T cells in the lymph nodes also expressed Il22bp (FIG. 2E). The expression levels were highest in CD44+CD4+ T cells, reaching levels similar to those in DCs. In the mouse colon, however, Il22bp was previously not detected in bulk populations of TCRb+ cells in steady-state conditions [10]. Additional analysis of purified CD4+ T cells from the colon was therefore performed. Il22bp was detected in colonic CD4+ T cells, but the expression level was low and close to the detection limit (FIG. 2E). In conclusion, both CD4+ T cells and DCs can produce IL-22BP in mice and humans.

T cell-specific up-regulation of IL-22BP in active IBD suggests a pathogenic role in intestinal inflammation. On the basis of these findings, the relevance of DC-versus T cell-derived IL-22BP in colitis was examined. To that end, the murine CD45RBhigh transfer colitis model was used, in which IL-22 mediates a protective function (10, 22). To discriminate between CD4+ T cell-derived and innate immune cell-derived IL-22BP, Il22bp−/− and Il22bp+/+ CD4+CD25−CD45RBhigh T cells were transferred into Rag1−/− and Rag1−/−Il22bp−/− mice. Transfer of wild-type T cells into Rag1−/− and Rag1−/−Il22bp−/− mice caused equally severe disease, characterized by weight loss and endoscopic and histological scores of colitis (FIG. 3, A to C). However, both Rag1−/− and Rag1−/−Il22bp−/− mice that received Il22bp-deficient T cells were largely protected from disease development (FIG. 3, A to C). Similar to the results observed in humans (FIG. 2C), the expression of IL-22BP by CD4+ T cells was increased in the murine colitis model (FIG. 8). T cells from Il22bp−/+ mice showed an intermediate level of Il22bp expression and caused an intermediate phenotype of colitis development (FIG. 9). The pathogenic effect of CD4+ T cell-derived IL-22BP was also confirmed in a bacterial-driven colitis model (FIG. 10). T cell-derived IL-22BP thus plays an important pathogenic role in multiple mouse IBD models.

In a next step the question was addressed whether the effect of IL-22BP is due to the free activity of IL-22 or to indirect mechanisms. Therefore, the transfer colitis experiment was performed in an IL-22-deficient environment. Il22−/− and Il22−/−Il 22bp−/− CD4+CD25−CD45RBhigh T cells were transferred into Rag1−/−Il22−/− mice. In this setting, both Il22−/− and Il22−/−Il22bp−/− CD4+CD25−CD45RBhigh T cells caused equally severe colitis (FIG. 11). As a control, Il22bp−/− CD4+CD25−CD45RBhigh T cells were transferred into Rag1−/− mice, which again were largely protected from colitis development (FIG. 11), indicating that IL-22BP aggravates colitis by blocking IL-22. In order to test whether Il22bp-deficient T cells have a cell-intrinsic defect, congenic wild-type and Il22bp-deficient CD4+CD25−CD45RBhigh T cells were cotransferred into Rag1−/− mice and the transferred T cells were analyzed upon colitis development. A significant difference in T cell numbers or cytokine production between wild-type and Il22bp-deficient T cells could not be found in this setting, arguing against a T cell-intrinsic defect of Il22bp-deficient T cells (FIG. 12). Taken together, the data show that IL-22 is sufficient to protect mice from effector T cell-mediated colitis in the absence of T cell-derived IL-22BP.

Anti-TNF-α therapy is the most effective treatment of IBD at present. It was therefore tested whether the efficacy of this therapy is linked to IL-22BP. A positive correlation between IL22BP and TNF-α expression in the intestine of IBD patients with active disease was found (P=0.004, correlation coefficient r=0.36; FIG. 4A). Genes encoding other cytokines, such as IL18, IL6, and IL23, did not correlate with IL22BP expression (FIG. 13). Moreover, IL22 also positively correlated with IL22BP (FIG. 13). However, Il22bp expression in the colon and lymph nodes of Il22−/− mice was not significantly reduced (FIG. 14), suggesting that IL-22 does not regulate IL-22BP. Therefore, the link between IL-22BP and anti-TNF-α therapy was further investigated.

CD4+ T cells and DCs were isolated from intestinal biopsy specimens obtained from IBD patients who were receiving anti-TNF-α therapy (adalimumab or infliximab) or other immune-modulating treatment in order to test whether anti-TNF-α treatment influences the expression of IL22BP. IL22BP expression was markedly reduced in CD4+ T cells of IBD patients who were responsive to anti-TNF-α treatment, compared with those of patients on other medications (FIG. 4B and table 4).

TABLE 4
Patient characteristics. Years are mean ± SD.

Treatment

| | | anti-TNFα | anti-TNFα |
|---|---|---|---|
| | Other | responder | non responder |
| | (n = 16) | (n = 5) | (n = 5) |
| Sex (F/M) | 5/11 | 0/5 | 2/3 |
| Age (years) | 44 ± 18.7 | 33 ± 11.8 | 48 ± 13.8 |
| Involvement UC | | | |
| left sided | 1 | 1 | 2 |
| distal | 3 | 1 | 1 |
| pancolitis | 3 | 0 | 0 |
| remission | 2 | 1 | 0 |
| Involvement CD | | | |
| ileum | 4 | 0 | 2 |
| colon | 1 | 1 | 0 |
| ileum + colon | 1 | 0 | 0 |
| remission | 1 | 1 | 0 |

This effect did not seem to be due to differences in disease activity [disease activity index (mean±SEM) for anti-TNF-α, 0.6±0.25; for other treatment, 0.3±0.13; P=0.29] and appeared to be specific to CD4+ T cells, because DCs did not show a significant down-regulation of IL22BP in IBD patients treated with anti-TNF-α (FIG. 4C). Furthermore, the expression of genes for other T cell signature cytokines and transcriptional regulators, such as IL17A, IL22, IFNg, IL5, FOXP3, TBX21, GATA3, and RORC, were not different between these groups (FIG. 4D and FIG. 15). These data argue against a general and broad effect of anti-TNF-α treatment on CD4+ T cells. However, it remained unclear whether TNF-α would regulate IL-22BP in a direct manner. It was found that IL22bp expression is not significantly reduced in Tnfr1- and Tnfr2-deficient T cells in the transfer colitis model, compared with that in wild-type controls (FIG. 16). Moreover, TNF-α did not induce IL22BP in T cells in vitro (FIG. 16). Taken together, these data show that TNF-α might regulate IL-22BP in an indirect manner that is as yet unknown.

Next it was tested whether anti-TNF-α therapy is simply inversely correlated with IL-22BP expression or whether the effect of this treatment is dependent on IL-22BP regulation, and thus on the protective effect of IL-22. In case of the latter, one would expect that this therapy would not work in an Il22-deficient environment. To test this, wild-type and Il22−/− CD4+CD25−CD45RBhigh T cells were transferred into Rag1−/− and Rag1−/−Il22−/− mice, respectively, and the mice were treated with anti-TNF-α upon colitis development. Anti-TNF-α treatment was not effective in the Il22-deficient environment (FIG. 17), but anti-TNF-α therapy significantly reduced colitis severity in the Il22-sufficient environment. In addition, Il22bp−/− CD4+CD25−CD45RBhigh T cells were transferred into Rag1−/−Il22bp−/− mice and the mice were treated with anti-TNF-α upon colitis development. As expected, these mice developed a mild colitis, which, however, was not further improved by anti-TNF-α therapy. To further support these data obtained in the murine system, CD4+ T cells isolated from the intestine of patients who did not respond to anti-TNF-α therapy were analyzed. T cell-derived IL-22BP was not down-regulated in these patients (FIG. 4B). Thus, the data show that at least one mechanism whereby anti-TNF-α therapy reduces disease activity is by down-regulating expression of IL-22BP.

### REFERENCES

1 N. Gagliani, B. Hu, S Huber, E. Elinav, R. A. Flavell, The fire within: Microbes in-flame tumors. Cell 157, 776-783 (2014). doi:10.1016/j.cell.2014.03.006
3 K. Wolk, E. Witte, U. Hoffmann, W. D. Doecke, S. Endesfelder, K. Asadullah, W. Sterry, H. D. Volk, B. M. Wittig, R. Sabat, IL-22 induces lipopolysaccharide-binding protein in hepatocytes: A potential systemic role of IL-22 in Crohn's disease. J. Immunol. 178, 5973-5981 (2007). doi:10.4049/jimmunol.178.9.5973
4 Basu, R. et al. Th22 cells are an important source of IL-22 for host protection against enteropathogenic bacteria. Immunity 37, 1061-1075, doi:10.1016/j.immuni.2012.08.024. (2012).
5. Kinnebrew, M. A. et al. Interleukin 23 Production by Intestinal CD103(+)CD11b(+) Dendritic Cells in Response to Bacterial Flagellin Enhances Mucosal Innate Immune Defense. Immunity 36, 276-287, doi: 10.1016/j.immuni.2011.12.011 (2012).
6 K. Sugimoto, A. Ogawa, E. Mizoguchi, Y. Shimomura, A. Andoh, A. K. Bhan, R. S. Blumberg, R. J. Xavier, A. Mizoguchi, IL-22 ameliorates intestinal inflammation in a mouse model of ulcerative colitis. J. Clin. Invest. 118, 534-544 (2008).
7 M. Kamanaka, S. Huber, L. A. Zenewicz, N. Gagliani, C. Rathinam, W. O'Connor Jr., Y. Y. Wan, S. Nakae, Y. Iwakura, L. Hao, R. A. Flavell, Memory/effector (CD45RBlo) CD4 T cells are controlled directly by IL-10 and cause IL-22-dependent intestinal pathology. J. Exp. Med. 208, 1027-1040 (2011). doi:10.1084/jem.20102149
8 L. A. Zenewicz, G. D. Yancopoulos, D. M. Valenzuela, A. J. Murphy, S. Stevens, R. A. Flavell, Innate and adaptive interleukin-22 protects mice from inflammatory bowel disease. Immunity 29, 947-957 (2008). doi:10.1016/j.immuni.2008.11.003
9 J. C. Martin, G. Bériou, M. Heslan, C. Chauvin, L. Utriainen, A. Aumeunier, C. L. Scott, A. Mowat, V. Cerovic, S. A. Houston, M. Leboeuf, F. X. Hubert, C. Hémont, M. Merad, S. Milling, R. Josien, Interleukin-22 binding protein (IL-22BP) is constitutively expressed by a subset of conventional dendritic cells and is strongly induced by retinoic acid. Mucosal Immunol. 7, 101-113 (2014). doi:10.1038/mi.2013.28
10 S. Huber, N. Gagliani, L. A. Zenewicz, F. J. Huber, L. Bosurgi, B. Hu, M. Hedl, W. Zhang, W. O'Connor Jr., A. J. Murphy, D. M. Valenzuela, G. D. Yancopoulos, C. J. Booth, J. H. Cho, W. Ouyang, C. Abraham, R. A. Flavell, IL-22BP is regulated by the inflammasome and modulates tumorigenesis in the intestine. Nature 491, 259-263 (2012).
   doi:10.1038/nature11535
11T. J. Ślebioda, Z. Kmieć, Tumour necrosis factor superfamily members in the pathogenesis of inflammatory bowel disease. Mediators Inflamm. 2014, 325129 (2014).
   doi:10.1155/2014/325129
12 A. Kaser, S. Zeissig, R. S. Blumberg, Inflammatory bowel disease. Annu. Rev. Immunol. 28, 573-621 (2010). doi:10.1146/annurev-immunol-030409-101225
13 S. Schmechel, A. Konrad, J. Diegelmann, J. Glas, M. Wetzke, E. Paschos, P. Lohse, B. Göke, S. Brand, Linking genetic susceptibility to Crohn's disease with Th17 cell function: IL-22 serum levels are increased in Crohn's disease and correlate with disease activity and IL23R genotype status. Inflamm. Bowel Dis. 14, 204-212 (2008). doi:10.1002/ibd.20315
14 C. C. Wei, T. W. Ho, W. G. Liang, G. Y. Chen, M. S. Chang, Cloning and characterization of mouse IL-22 binding protein. Genes Immun. 4, 204-211 (2003).
   doi:10.1038/sj.gene.6363947
15 S. V. Kotenko, L. S. Izotova, O. V. Mirochnitchenko, E. Esterova, H. Dickensheets, R. P. Donnelly, S. Pestka, Identification, cloning, and characterization of a novel soluble receptor that binds IL-22 and neutralizes its activity. J. Immunol. 166, 7096-7103 (2001).
   doi:10.4049/jimmuno1.166.12.7096
16 W. Xu, S. R. Presnell, J. Parrish-Novak, W. Kindsvogel, S. Jaspers, Z. Chen, S. R. Dillon, Z. Gao, T. Gilbert, K. Madden, S. Schlutsmeyer, L. Yao, T. E. Whitmore, Y. Chandrasekher, F. J. Grant, M. Maurer, L. Jelinek, H. Storey, T. Brender, A. Hammond, S. Topouzis, C. H. Clegg, D. C. Foster, A soluble class II cytokine receptor, IL-22RA2, is a naturally occurring IL-22 antagonist. Proc. Natl. Acad. Sci. U.S.A. 98, 9511-9516 (2001).
   doi:10.1073/pnas.171303198
17 L. Dumoutier, D. Lejeune, D. Colau, J. C. Renauld, Cloning and characterization of IL-22 binding protein, a natural antagonist of IL-10-related T cell-derived inducible factor/IL-22. J. Immunol. 166, 7090-7095 (2001). doi:10.4049/jimmuno1.166.12.7090
18 B. C. Jones, N. J. Logsdon, M. R. Walter, Structure of IL-22 bound to its high-affinity IL-22R1 chain. Structure 16, 1333-1344 (2008). Medline doi:10.1016/j.str.2008.06.005
19 S. Kirchberger, D. J. Royston, O. Boulard, E. Thornton, F. Franchini, R. L. Szabady, O. Harrison, F. Powrie, Innate lymphoid cells sustain colon cancer through production of inter-leukin-22 in a mouse model. J. Exp. Med. 210, 917-931 (2013). doi:10.1084/jem.20122308
20 I. Kryczek, Y. Lin, N. Nagarsheth, D. Peng, L. Zhao, E. Zhao, L. Vatan, W. Szeliga, Y. Dou, S. Owens, W. Zgodzinski, M. Majewski, G. Wallner, J. Fang, E. Huang, W. Zou, IL-22+CD4+ T cells promote colorectal cancer sternness via STAT3 transcription factor activation and induction of the methyltransferase DOT1L. Immunity 40, 772-784 (2014).
   doi:10.1016/j.immuni.2014.03.010
21 K. Moriwaki, S. Balaji, T. McQuade, N. Malhotra, J. Kang, F. K. Chan, The necroptosis adaptor RIPK3 promotes injury-induced cytokine expression and tissue repair. Immunity 41, 567-578 (2014). doi:10.1016/j.immuni.2014.09.016
22 P. B. Watchmaker, K. Lahl, M. Lee, D. Baumjohann, J. Morton, S. J. Kim, R. Zeng, A. Dent, K. M. Ansel, B. Diamond, H. Hadeiba, E. C. Butcher, Comparative transcriptional and functional profiling defines conserved programs of intestinal DC differentiation in humans and mice. Nat. Immunol. 15, 98-108 (2014). doi:10.1038/ni.2768
23 F. Powrie, R. Correa-Oliveira, S. Mauze, R. L. Coffman, Regulatory interactions between CD45RBhighand CD45RBlowCD4+ T cells are important for the balance between protective and pathogenic cell-mediated immunity. J. Exp. Med. 179, 589-600 (1994).
   doi:10.1084/jem.179.2.589
24 C. Papi, F. Fasci-Spurio, F. Rogai, A. Settesoldi, G. Margagnoni, V. Annese, Mucosal healing in inflammatory bowel disease: Treatment efficacy and predictive factors. Dig. Liver Dis. 45, 978-985 (2013). doi:10.1016/j.d1d.2013.07.006
25 Livak KJ, Schmittgen TD. Analysis of relative gene expression data using real-time quantitative PCR and the 2(-Delta Delta C(T)) Method. Methods. 25:402-408.
26. J. C. Martin, G. Bériou, M. Heslan, C. Bossard, A. Jarry, A. Abidi, P. Hulin, S. Ménoret, R. Thinard, I. Anegon, C. Jacqueline, B. Lardeux, F. Halary, J. C. Renauld, A. Bourreille, R. Josien, IL-22BP is produced by eosinophils in human gut and blocks IL-22 protective actions during colitis. Mucosal Immunol. 9, 539-549 (2016). doi:10.1038/mi.2015.83
27. C. Becker, M. C. Fantini, M. F. Neurath, High resolution colonoscopy in live mice. Nat. Protoc. 1, 2900-2904 (2007). doi:10.1038/nprot.2006.446
28. W. O'Connor Jr., M. Kamanaka, C. J. Booth, T. Town, S. Nakae, Y. Iwakura, J. K. Kolls, R. A. Flavell, A protective function for interleukin 17A in T cell-mediated intestinal inflammation. Nat. Immunol. 10, 603-609 (2009). doi:10.1038/ni.1736
29. J. Schrader, T. T. Gordon-Walker, R. L. Aucott, M. van Deemter, A. Quaas, S. Walsh, D. Benten, S. J. Forbes, R. G. Wells, J. P. Iredale, Matrix stiffness modulates proliferation, chemotherapeutic response, and dormancy in hepatocellular carcinoma cells. Hepatology 53, 1192-1205 (2011).

1. A method for determining the expression level of Inter leukin-22 binding protein, IL-22BP, in a subject, the method comprising:
a. obtaining a biological sample from the subject, and
b. determining the expression level of IL-22BP by contacting the biological sample with an antibody specific for IL-22BP and detecting the level of binding between IL-22BP and the antibody.

2. The method according to claim 1, comprising determining the expression level of IL-22BP in a first biological sample obtained from the subject at a first date and in a second biological sample obtained from the subject at a later second date, and comparing the IL-22BP expression levels at the first and second date.

3. The method according to claim 2, wherein said determining is part of an anti-TNF-alpha-treatment, and wherein a) the first biological sample is obtained at a date before the treatment and the second biological sample is obtained at a date during or after the treatment, or b) the first biological sample is obtained at a date during the treatment and the second biological sample is obtained at a later date during the treatment or at a date after the treatment.

4. The method according to claim 3, wherein the anti-TNF-alpha-treatment is an anti-TNF-alpha-treatment of an inflammatory bowel disease.

5. The method according to claim 4, wherein the biological sample is a biopsy specimen taken from the intestine of the subject.

6. The method according to claim 1, wherein the expression level of T-cell derived IL-22BP is determined.

7. The method according to claim 4, wherein the biological sample is a biopsy specimen taken from an inflamed area of the large intestine or small intestine of the subject.

8. A method for monitoring the effectiveness of an anti-TNF-alpha-treatment in a subject, said method comprising:
a. obtaining a biological sample from the subject, and
b. determining the expression level of IL-22BP by contacting the biological sample with mouse derived antibody specific for IL-22BP and detecting the level of binding between IL-22BP and the mouse derived antibody specific for IL-22BP.

9. The method according to claim 8, wherein the antibody is mouse derived anti-human IL-22BP antibody clone 87554.

10. A method for monitoring and treating Inflamatory Bowel Disease (IBD) with an anti-TNF-alpha treatment in a subject comprising
a. obtaining a biological sample from the subject,
b. determining the expression level of IL-22BP by contacting the biological sample with antibody specific for IL-22BP and detecting the level of binding between IL-22BP and the antibody specific for IL-22BP, and
c. adjusting the anti-TNF-alpha treatment as a result of the determined expression level of IL-22BP.

11. The method according to claim 10, wherein said adjusting comprises administering an anti-TNA-alpha agent, and
if IL-22BP level is high,increasing the dose of an anti-TNF-alpha agent or administering an alternative anti-TNF-alpha agent having a stronger effect than a previously administered agent, and
if IL-22BP level is low, decreasing the dose of an anti-TNF-alpha agent or administering an alternative anti-TNF-alpha agent having a weaker effect than a previously administered agent.
